(19) United States
(12) Patent Application Publication
(10) Pub. No.: US 2025/0388951 A1
(43) Pub. Date: Dec. 25, 2025

(54) MONOCLONAL POLONY GENERATION USING NUCLEIC ACID SUPRAMOLECULAR STRUCTURES

(71) Applicant: SOMALOGIC OPERATING CO., INC., Boulder, CO (US)

(72) Inventor: Ashwin GOPINATH, La Jolla, CA (US)

(21) Appl. No.: 18/877,608

(22) PCT Filed: Jun. 16, 2023
(86) PCT No.: PCT/US2023/068609

§ 371 Date: Dec. 20, 2024

Related U.S. Application Data
(60) Provisional application No. 63/355,744, filed on Jun. 27, 2022.

(51) Int. Cl. C12Q 1/6806; C08J 3/075; C08J 3/24; C12Q 1/6813; C12Q 1/6844; C12Q 1/6876

(52) U.S. Cl. CPC C12Q 1/6806; C08J 3/075; C08J 3/24; C12Q 1/6813; C12Q 1/6844; C12Q 1/6876

(57) ABSTRACT

Provided herein are structures and methods for generating monoclonal clusters of a target oligonucleotide in a solution using a first nucleic acid supramolecular structure. In some cases, the target oligonucleotide is released from a second nucleic acid supramolecular structure. In some cases. the first and second nucleic acid supramolecular structure independently comprise DNA origami. In certain cases, the nucleic acid supramolecular structures may be coated in a hydrogel matrix. In other cases, the hydrogel matrix may be omitted. The monoclonal clusters created in solution using the disclosed structures and techniques may be immobilized on a substrate to facilitate subsequent processes performed on the monoclonal clusters.

### BACKGROUND

The ability to manipulate and process the molecular components of biological systems has rapidly expanded over the last few decades, including the ability to sequence the molecular nucleotide strands coding the information that is transcribed and translated into functioning proteins. Indeed, the efficiency and capacity for performing such sequencing operations has steadily increased while, correspondingly, the costs have decreased. For example, it is currently possible to sequence the entire human genome consisting of approximately 4 billion base pairs in under a day and at a price point of approximately $100.

The typical workflow for next-generation sequencing (NGS) technologies, except for single-molecule sequencing platforms, typically involves library generation, colony formation, sequencing, and analysis. Library generation is the process by which a genomic DNA sample (or other nucleic acid sample) is fragmented and specific adaptor strands are attached for downstream processing. Following the library generation, colony formation is the step where many copies of each single library fragment is made on a bead or in specific region of a solid support. The most widely used approach for colony formation is the polymerase colony (polony) technology approach, which involves generating monoclonal clusters by performing Polymerase Chain Reaction (PCR) after a library fragment is attached to a bead, hydrogel matrix or solid support with primers bound to it. After colony generation, the complementary section of the library fragment is extended cycle-by-cycle using reversible terminators and the extended nucleotides (A, C, G, or T) in each cycle are identified using optical-based scanning platforms and techniques or using electrical readout based technologies. During the analysis step, the signals from different clusters are analyzed to reconstruct the underlying sequences.

Over the last two decades there has been significant improvement, both in terms of ease and yield, in the methods employed for library preparation, sequencing, and analysis. However, neither the ease, nor the yield of colony generation has been improved significantly due to the inherent randomness associated with the binding of a library fragment onto a bead or a solid support.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a process flow illustrating steps in the creation of a hydrogel-coated particle, in accordance with aspects of the present disclosure;

FIG. 2 visually illustrates aspects of the creation of a supramolecular structure, in accordance with aspects of the present disclosure;

FIG. 3 visually illustrates aspects of coating a supramolecular structure with a hydrogel matrix, in accordance with aspects of the present disclosure;

FIG. 4 visually illustrates aspects of attaching primers to a hydrogel matrix, in accordance with aspects of the present disclosure;

FIG. 5 depicts an exemplary depiction of a second nucleic acid supramolecular structure and the related subcomponents, in accordance with aspects of the present disclosure;

FIG. 6 provides an exemplary depiction of a supramolecular structure in an unstable state before and after being subject to a trigger (e.g., interaction with a deconstructor molecule), in accordance with aspects of the present disclosure;

FIG. 7 provides an exemplary depiction of a supramolecular structure in a stable state before and after being subject to a trigger (e.g., interaction with a deconstructor molecule), in accordance with aspects of the present disclosure;

FIG. 8 depicts a process flow illustrating steps in the use of a hydrogel-coated particle to generate monoclonal clusters, in accordance with aspects of the present disclosure;

FIG. 9 visually illustrates aspects of amplifying an oligonucleotide attached to a hydrogel-coated particle, in accordance with aspects of the present disclosure;

FIG. 10 visually illustrates aspects of immobilizing monoclonal clusters on a substrate, in accordance with aspects of the present disclosure;

FIG. 11 depicts a process flow illustrating steps in the creation of a capture and amplification structure, in accordance with aspects of the present disclosure;

FIG. 12 visually illustrates aspects of the creation of a supramolecular structure, in accordance with further aspects of the present disclosure;

FIG. 13 depicts a process flow illustrating steps in the use of a capture and amplification structure to generate monoclonal clusters, in accordance with aspects of the present disclosure; and

FIG. 14 shows a block diagram of an example processing system according to embodiments of the present disclosure.

### SUMMARY

The present disclosure generally relates to systems, structures and methods for producing monoclonal clusters of nucleic acid fragments (e.g., oligonucleotides of deoxyribonucleic acid (DNA) or ribonucleic acid (RNA)) directly in a solution phase, without the need for compartmentalization or spatial organization. In certain embodiments, the monoclonal clusters are formed on nucleic acid supramolecular structures (such as DNA origami structures) by capturing individual, specific oligonucleotides (e.g., having a specific nucleic acid sequence within the oligonucleotide) and enzymatically amplifying the captured oligonucleotides. In certain such implementations, the nucleic acid supramolecular structures are coated in a hydrogel matrix, such as a thermostable hydrogel matrix. The monoclonal clusters generated in solution are also designed to facilitate precise organization on a substrate (e.g., a planar substrate).

As discussed herein, in certain embodiments, each supramolecular structure includes a core structure, which in turn comprises a plurality of core molecules. In certain implementations each core structure is a nanostructure. The plurality of core molecules for each core structure may be arranged into a pre-defined shape or geometry and/or may have a prescribed molecular weight. In certain such embodiments, the pre-defined shape or geometry is configured to limit or prevent cross-reactivity with other supramolecular structures. In some embodiments, the plurality of core molecules for each core structure comprises one or more nucleic acid strands, one or more branched nucleic acids, one or more peptides, one or more small molecules, or combinations thereof. In some embodiments, for any method disclosed herein, each core structure independently comprises a scaffolded deoxyribonucleic acid (DNA) origami, a scaffolded ribonucleic acid (RNA) origami, a scaffolded hybrid DNA: RNA origami, a single-stranded DNA tile structure, a multi-stranded DNA tile structure, a single-stranded RNA origami, a multi-stranded RNA tile structure, hierarchically composed DNA or RNA origami with multiple scaffolds, a peptide structure, or combinations thereof.

By way of further example, in a practical implementation a DNA origami may be created and coated with a thermostable hydrogel matrix. The hydrogel-coated DNA origami may be modified with one or more surface modifications. One such modification may be the inclusion or addition of a first chemical group (e.g., a first chemically reactive group) that is designed or selected to capture a specific oligonucleotide (i.e., an oligonucleotide have a specific nucleic acid sequence within the oligonucleotide) from solution. As will be appreciated, different hydrogel-coated particles may be modified with chemically reactive groups having different specificities, such that the different or various modified hydrogel-coated particles may have different oligonucleotides for which they have specificity. More generally, different supramolecular structures of a pool of supramolecular structures comprise different first chemically reactive groups with different binding affinity for different oligonucleotide sequences within a sample.

Another modification may be the addition of pairs of DNA primers to the hydrogel-coated particle. The primers in this example enable local amplification of the oligonucleotide for which the hydrogel-coated particle has specificity once such oligonucleotides are captured. A further modification in this context may be the addition of a second chemical group (e.g., a second chemically reactive group) to the surface of the hydrogel-coated particle that, in one example, enables immobilization of the hydrogel-coated particle to a substrate, such as a single-molecule array of capture sites. For example, the second chemical group may have affinity to a target or specific binding molecule present on the substrate so as to form a binding attachment when in the presence of the substrate. As used herein, the substrate may comprise a solid support, solid substrate, a polymer matrix, or one or more beads.

In certain instances, a unique identifier sequence (e.g., a tag or barcode, such as a nucleic acid having a unique barcode sequence) may be provided as part of (or in place of) the second chemical group, such as part of the molecular chain forming the second chemically reactive group or as a branch off of such a molecular chain. The unique identifier may, directly or indirectly, be indicative of a specific oligonucleotide corresponding to the capture affinity of the first chemically reactive group associated with a respective hydrogel-coated particle. In some embodiments, each unique identifier sequence (e.g., barcode) provides a DNA signal or initiator signal corresponding to the respective specific oligonucleotide. In some embodiments, the unique identifier sequence is analyzed using genotyping, qPCR, sequencing, or combinations thereof.

In practice, the size of the supramolecular structure may be useful in the context of a substrate having permissive binding sites. In particular, as discussed herein, each supramolecular structure, whether hydrogel-coated or not, captures a specific oligonucleotide. The space restrictions arising due to the size of the supramolecular structure may limit or otherwise restrict binding events at a given substrate binding site, such as to a one-to-one relationship, effectively associating each binding site of the substrate with a specific oligonucleotide.

With this high-level overview in mind, in operation a hydrogel-coated particle (e.g., a hydrogel-coated nucleic acid supramolecular structure, such as a DNA origami) as discussed herein may be used to capture oligonucleotides (e.g., specific oligonucleotides for each hydrogel-coated particle) in a solution. The captured oligonucleotides may undergo local amplification via the pairs of primers attached to the hydrogel-coated particles to yield particles having multiple copies of the captured oligonucleotide. In one implementation, the hydrogel-coated particle may then be immobilized on a substrate (such as via a binding interaction between the second chemically reactive group and sites on the substrate). By way of example, the substrate can be a single-molecule array of capture sites, which may be used in downstream processing steps or operations, such as sequencing operations.

Provided herein in one embodiment is a method for forming a monoclonal cluster of a target oligonucleotide on a hydrogel particle. In some embodiments, the method comprises incubating a first nucleic acid supramolecular structure, comprising a first chemically reactive group, with a plurality of polymer molecules to form a hydrogel matrix around the first nucleic acid supramolecular structure, providing a crosslinking agent to the hydrogel matrix around the first nucleic acid supramolecular structure to form the hydrogel particle, incubating the hydrogel particle with a second nucleic acid supramolecular structure, comprising the target oligonucleotide, sufficiently enough to facilitate capture of the target oligonucleotide by the first chemically reactive group, and amplifying the target oligonucleotide thereby producing copies of the target oligonucleotide attached to the hydrogel particle. In some embodiments, the first nucleic acid supramolecular structure is configured to be sufficiently thermostable to support a nucleic acid amplification.

In some embodiments, the first chemically reactive group has an affinity to the target oligonucleotide. In some embodiments, the second supramolecular structure is configured to release the target oligonucleotide in absence of an analyte molecule. In some embodiments, the first nucleic acid supramolecular structure further comprises the first core structure and a second chemically reactive group. In some embodiments, the first chemically reactive group is linked to the first core structure at a first location or a first set of locations. In some embodiments, the second chemically reactive group is linked to the first core structure at a second location, wherein the second location is spatially separated from the first location or the first set of locations. In some embodiments, the second chemically reactive group has an affinity to a corresponding binding molecule associated with a substrate. In some embodiments, the first location or the first set of locations and the second location are separated spatially enough to avoid cross-reactivity between the first chemically reactive group and the second chemically reactive group.

In some embodiments, the plurality of polymer molecules comprises a block copolymer. In some embodiments, the block copolymer comprises one or more charged regions, one or more uncharged regions, a free terminal associated with the uncharged region, and one or more reactive molecules attached to the free terminal. In some embodiments, the one or more uncharged regions comprises a neutral polymer. In some embodiments, the neutral polymer comprises polyethylene glycol (PEG). In some embodiments, the one or more charged region comprises a charged polymer. In some embodiments, the charged polymer comprises poly-L-lysine (PLL) or poly-acrylic acid (PAA).

In some embodiments, the second nucleic acid supramolecular structure further comprises a second core structure and a capture molecule, wherein the capture molecule is linked to the second core structure at a third location. In some embodiments, the target oligonucleotide is linked to the second core structure at a fourth location, wherein the fourth location is spatially separate from the third location. In some embodiments, the target oligonucleotide is configured to be released from the second core structure by providing a trigger to cleave the link between the target oligonucleotide and the second core structure in absence of the analyte molecule. In some embodiments, the target oligonucleotide remains to be bound to the second core structure through binding to the analyte molecule after providing the trigger to cleave the link between the target oligonucleotide and the second core structure in presence of the analyte molecule, wherein the analyte molecule also binds to the capture molecule at the third location of the second core structure.

In some embodiments, the method further comprises detecting the analyte molecule based on whether copies of the target oligonucleotide are formed on the hydrogel particle. In some embodiments, the analyte molecule comprises a protein, a peptide, a peptide fragment, a lipid, a DNA, a RNA, an organic molecule, an inorganic molecule, complexes thereof, or any combinations thereof. In some embodiments, the trigger comprises DNA, RNA, a peptide, a small organic molecule, or combinations thereof. In some embodiments, the analyte molecule binds to the capture molecule through a chemical bond and/or binds to the target oligonucleotide through a chemical bond. In some embodiments, the capture molecule comprises a protein, a peptide, an antibody, an aptamer (RNA and DNA), a fluorophore, a darpin, a catalyst, a polymerization initiator, a polymer like PEG, or combinations thereof. In some embodiments, the crosslinking agent comprises glutaraldehyde, bis(sulfosuccinimidyl)suberate (BS3), di-tert-butyl peroxide (DTBP), or combinations thereof.

In some embodiments, nucleic acid primers are linked to the hydrogel matrix. In some embodiments, the nucleic acid primers facilitate amplification of the target oligonucleotide when the target oligonucleotide is captured by the first reactive group. In some embodiments, the first reactive group and the nucleic acid primers are extruded out of the hydrogel matrix. In some embodiments, the first nucleic acid supramolecular structure is configured to be thermostable via UV irradiation. In some embodiments, the second nucleic acid supramolecular structure is configured to be sufficiently thermostable to support a nucleic acid amplification. In some embodiments, the first core structure and the second core structure independently comprise a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, an enzymatically synthesized nucleic acid structure, a nucleic acid structure created by tile assembly, or combinations thereof.

In some embodiments, amplifying the target oligonucleotide comprises a local amplification. In some embodiments, amplifying the target oligonucleotide comprises using the nucleic acid primers. In some embodiments, a first adaptor strand and a second adaptor strand are independently ligated to each end of the target oligonucleotide, wherein the first adaptor strand comprises sequence complementary to one nucleic acid primer of the pair of nucleic acid primer and the second adaptor strand comprises sequence complementary to the other nucleic acid primer of the pair of nucleic acid primer. In some embodiments, the first adaptor strand comprises a reactive group, wherein the reactive group is configured to bind to the first chemically reactive group. In some embodiments, incubating the hydrogel particle and the second nucleic acid supramolecular structure is performed in a denaturing condition to avoid interaction between the adaptors and the nucleic acid primers. In some embodiments, the method further comprises, prior to amplifying the target oligonucleotide, exchanging the denaturing condition to a non-denaturing condition to allow interaction between the adaptors and the nucleic acid primers. In some embodiments, amplifying the target oligonucleotide further comprises adding enzymes. In some embodiments, amplifying the target oligonucleotide further comprises using a thermocycler.

In some embodiments, the hydrogel particle is immobilized on a substrate. In some embodiments, the hydrogel particle is immobilized on the substrate by a binding between the second chemically reactive group disposed on the hydrogel particle and a corresponding binding molecule attached to the substrate, wherein the second chemically reactive group has a binding affinity to the corresponding binding molecule. In some embodiments, the second chemically reactive group comprises a thiol reactive group, an azide reactive group, an amine reactive group, or a carboxyl reactive group. In some embodiments, the substrate comprises a single-molecule array. In some embodiments, the single-molecule array comprises a second nucleic acid supramolecular structure, each second nucleic supramolecular structure comprising the corresponding binding molecule.

The present disclosure also relates to a method for detecting an analyte molecule. In some embodiments, the method comprises incubating a first nucleic acid supramolecular structure, comprising a first chemically reactive group, with a plurality of polymer molecules to form a hydrogel matrix around the first nucleic acid supramolecular structure, providing a crosslinking agent to the hydrogel matrix around the first nucleic acid supramolecular structure to form the hydrogel particle, incubating the hydrogel particle with a second nucleic acid supramolecular structure, comprising the target oligonucleotide, to facilitate capture of the target oligonucleotide by the first chemically reactive group, providing conditions for amplification of the target oligonucleotide, and detecting the analyte molecule based on lack of amplification from the previous step. In some embodiments, the first nucleic acid supramolecular structure is configured to be sufficiently thermostable to support a nucleic acid amplification. In some embodiments, the first chemically reactive group has an affinity to a target oligonucleotide.

In some embodiments, the second supramolecular structure is configured to release the target oligonucleotide in absence of an analyte molecule. In some embodiments, the first nucleic acid supramolecular structure further comprises the first core structure and a second chemically reactive group. In some embodiments, the first chemically reactive group is linked to the first core structure at a first location or a first set of locations. In some embodiments, the second chemically reactive group is linked to the first core structure at a second location, wherein the second location is spatially separated from the first location or the first set of locations. In some embodiments, the second chemically reactive group has an affinity to a corresponding binding molecule associated with a substrate. In some embodiments, the first location or the first set of locations and the second location are separated spatially enough to avoid cross-reactivity between the first chemically reactive group and the second chemically reactive group.

In some embodiments, the plurality of polymer molecules comprises a block copolymer. In some embodiments, the block copolymer comprises one or more charged regions, one or more uncharged regions, a free terminal associated with the uncharged region, and one or more reactive molecules attached to the free terminal. In some embodiments, the one or more uncharged regions comprises a neutral polymer. In some embodiments, the neutral polymer comprises polyethylene glycol (PEG). In some embodiments, the one or more charged region comprises a charged polymer. In some embodiments, the charged polymer comprises poly-L-lysine (PLL) or poly-acrylic acid (PAA).

In some embodiments, the second nucleic acid supramolecular structure further comprises a second core structure and a capture molecule, wherein the capture molecule is linked to the second core structure at a third location. In some embodiments, the target oligonucleotide is linked to the second core structure at a fourth location, wherein the fourth location is spatially separate from the third location. In some embodiments, the target oligonucleotide is configured to be released from the second core structure by providing a trigger to cleave the link between the target oligonucleotide and the second core structure in absence of the analyte molecule. In some embodiments, the target oligonucleotide remains to be bound to the second core structure through binding to the analyte molecule after providing the trigger to cleave the link between the target oligonucleotide and the second core structure in presence of the analyte molecule, wherein the analyte molecule also binds to the capture molecule at the third location of the second core structure.

In some embodiments, the method further comprises detecting the analyte molecule based on whether copies of the target oligonucleotide are formed on the hydrogel particle. In some embodiments, the analyte molecule comprises a protein, a peptide, a peptide fragment, a lipid, a DNA, a RNA, an organic molecule, an inorganic molecule, complexes thereof, or any combinations thereof. In some embodiments, the trigger comprises DNA, RNA, a peptide, a small organic molecule, or combinations thereof.

In some embodiments, the analyte molecule binds to the capture molecule through a chemical bond and/or binds to the target oligonucleotide through a chemical bond. In some embodiments, the capture molecule comprises a protein, a peptide, an antibody, an aptamer (RNA and DNA), a fluorophore, a darpin, a catalyst, a polymerization initiator, a polymer like PEG, or combinations thereof. In some embodiments, the crosslinking agent comprises glutaraldehyde, bis(sulfosuccinimidyl)suberate (BS3), di-tert-butyl peroxide (DTBP), or combinations thereof. In some embodiments, nucleic acid primers are linked to the hydrogel matrix.

In some embodiments, the nucleic acid primers facilitate amplification of the target oligonucleotide when the target oligonucleotide is captured by the first reactive group. In some embodiments, the first reactive group and the nucleic acid primers are extruded out of the hydrogel matrix. In some embodiments, the first nucleic acid supramolecular structure is configured to be thermostable via UV irradiation.

In some embodiments, the first core structure and the second core structure independently comprise a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, an enzymatically synthesized nucleic acid structure, a nucleic acid structure created by tile assembly, or combinations thereof.

In some embodiments, amplifying the target oligonucleotide comprises a local amplification. In some embodiments, amplifying the target oligonucleotide comprises using the nucleic acid primers. In some embodiments, a first adaptor strand and a second adaptor strand are independently ligated to each end of the target oligonucleotide, wherein the first adaptor strand comprises sequence complementary to one nucleic acid primer of the pair of nucleic acid primer and the second adaptor strand comprises sequence complementary to the other nucleic acid primer of the pair of nucleic acid primer. In some embodiments, the first adaptor strand comprises a reactive group, wherein the reactive group is configured to bind to the first chemically reactive group. In some embodiments, incubating the hydrogel particle and the second nucleic acid supramolecular structure is performed in a denaturing condition to avoid interaction between the adaptors and the nucleic acid primers.

In some embodiments, the method further comprises, prior to amplifying the target oligonucleotide, exchanging the denaturing condition to a non-denaturing condition to allow interaction between the adaptors and the nucleic acid primers. In some embodiments, amplifying the target oligonucleotide further comprises adding enzymes.

In some embodiments, amplifying the target oligonucleotide further comprises using a thermocycler.

In some embodiments, the hydrogel particle is immobilized on a substrate. In some embodiments, the hydrogel particle is immobilized on the substrate by a binding between the second chemically reactive group disposed on the hydrogel particle and a corresponding binding molecule attached to the substrate, wherein the second chemically reactive group has a binding affinity to the corresponding binding molecule. In some embodiments, the second chemically reactive group comprises a thiol reactive group, an azide reactive group, an amine reactive group, or a carboxyl reactive group. In some embodiments, the substrate comprises a single-molecule array. In some embodiments, wherein the single-molecule array comprises a second nucleic acid supramolecular structure, each second nucleic supramolecular structure comprising the corresponding binding molecule.

With respect to a sample processed using the techniques discussed herein, such a sample will be suitable for processing for selective oligonucleotide capture in a solution phase and subsequent amplification to form monoclonal clusters in solution. The sample in question may be a biological sample. In some embodiments, the sample comprises an aqueous solution comprising an oligonucleotide of interest or a variety of differing oligonucleotides, some or all of which may be of interest. The sample may comprise or may be derived from a tissue biopsy, blood, blood plasma, urine, saliva, a tear, cerebrospinal fluid, extracellular fluid, cultures cells, culture media, discarded tissue, plant matter, synthetic proteins, bacterial, viral samples, fungal tissue, or combinations thereof. By way of example, the sample may be isolated from a primary source such as cells, tissue, bodily fluids (e.g., blood), environmental samples, or combinations thereof, with or without purification. In embodiments where cells are involved in sample preparation, the cells may be lysed using a mechanical process or other cell lysis methods (e.g., lysis buffer). The sample may be filtered using a mechanical process (e.g., centrifugation), micron filtration, chromatography columns, other filtration methods, or combinations thereof. Further. the sample may or may not be treated with one or more enzymes to remove one or more nucleic acids or one or more proteins. In some embodiments, the sample comprises denatured nucleic acids or degraded nucleic acid fragments. In certain implementations the sample is collected from one or more individual persons, one or more animals, one or more plants, or combinations thereof. By way of example, the sample may be collected from an individual person (e.g., a patient of subject), animal and/or plant having a disease or disorder that comprises an infectious disease, an immune disorder, a cancer, a genetic disease, a degenerative disease, a lifestyle disease, an injury, a rare disease, an age-related disease, or combinations thereof.

The sample may be processed to release the oligonucleotides from cells or to otherwise prepare the sample for analysis prior to contacting the sample with the supramolecular structures in solution as provided herein.

### DETAILED DESCRIPTION

Throughout this application, various embodiments of this disclosure may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “about” and “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, the terms can mean a range of up to 20%, up to 10%, up to 5%, or up to 1% of a given value. Alternatively, the terms can mean within an order of magnitude, preferably within 5-fold, and more preferably within 2-fold, of a value. As used herein, the term “analytes” and “analyte molecules” are used interchangeably.

As used herein, the terms “binding,” “bound,” and “interaction” are used interchangeably, and generally refer to a non-covalent interaction between macromolecules (e.g., between a protein and a nucleic acid). While in a state of non-covalent interaction, the macromolecules are said to be “associated,” “interacting,” or “binding” (e.g., when a molecule X is said to interact with a molecule Y, it is meant the molecule X binds to molecule Y in a non-covalent manner).

As used herein, the terms “attaching,” “linking,” “linkage,” and “link” are used interchangeably, and generally refer to connecting one entity to another. For example, oligomers and primers may be attached to the surface of a capture site. With respect to attaching mechanisms, methods contemplated include such attachment means as ligating, non-covalent bonding, binding of biotin moieties such as biotinylated primers, amplicons, and probes to streptavidin, etc. A capture molecule may for example be attached directly to a supramolecular structure (e.g., via a covalent bond, a biotin-streptavidin bond, a DNA oligonucleotide linker, or a polymer linker) or indirectly (e.g., via linkage to an anchor strand, e.g., by conjugation or through a linker such as a capture strand).

As used herein, the term “are linked together” in some embodiments refers to enabling the formation of a chemical bond. In some embodiments, as used herein, a chemical bond refers to a lasting attraction between atoms, ions or molecules. The bond includes covalent bonds, ionic bonds, hydrogen bonds, van der Waals interactions, or any combination thereof. In some embodiments, the term “are linked together” refers to hybridization of nucleic acids which is the process of combining two complementary single-stranded DNA or RNA molecules and allowing them to form a single double-stranded molecule through base pairing.

As used herein, the term “nucleic acid origami” generally refers to a nucleic acid construct comprising an engineered tertiary (e.g., folding and relative orientation of secondary structures) or quaternary structure (e.g., hybridization between strands that are not covalently linked to each other) in addition to the naturally occurring secondary structure (e.g., helical structure) of nucleic acid(s). A nucleic acid origami may include DNA, RNA, PNA, modified or non-natural nucleic acids, or combinations thereof. A nucleic acid origami can include a scaffold strand. The scaffold strand can be circular (i.e., lacking a 5′ end and 3′ end) or linear (i.e., having a 5′ end and/or a 3′ end). A nucleic acid origami may include a plurality of oligonucleotides that hybridize via sequence complementarity to produce the engineered structuring of the origami particle. For example, the oligonucleotides can hybridize to a scaffold strand and/or to other oligonucleotides. A nucleic acid origami may comprise sections of single-stranded or double-stranded nucleic acid, or combinations thereof. Exemplary nucleic acid origami structures may include nanotubes, nanowires, cages, tiles, nanospheres, blocks, and combinations thereof.

The term “crosslinker” used herein refers to a crosslinker that assists formation (and stabilization) of hydrogel around a DNA nanostructure. In some embodiments, the crosslinker comprises, but not limited to, glutaraldehyde, bis(sulfosuccinimidyl)suberate (BS3), di-tert-butyl peroxide (DTBP), or combinations thereof. In some cases, glutaraldehyde may crosslink (and stabilize) the lysine residues in PLL-PEG matrix that is formed around the DNA nanostructure, initially through electrostatic interaction. In some embodiments, the term “crosslinker” used herein refers to a crosslinker that interacts directly with terminal groups on the 3′ and 5′ of oligos that form the DNA origami. By way of example, all staple strands of a DNA origami, which have an amine residue on both the 3′ and 5′ end, may be crosslinked using gluteraldehyde (or formaldehyde). In another cases, as used herein, the term “crosslinker” refers to a crosslinker that crosslinks thymines by UV irradiation. In some embodiments, a DNA origami with all staple strands having a poly-T extension (1-5 based long) on both 3′ and 5′ end may have the thymines crosslinked by UV irradiation. As used herein, the term “crosslinker” may also refer to a chemical crosslinker that directly crosslinks DNA itself. In some embodiments, the crosslinker comprises cisplatin or UV assisted crosslinker (e.g., psoralen).

Disclosed herein are structures and methods for forming a monoclonal cluster of a target oligonucleotide on a hydrogel particle in a solution using hydrogel matrix coated a first supramolecular structure. In some embodiments, the hydrogel matrix and the first supramolecular structure may be sufficiently thermostable to support a nucleic acid amplification. In some embodiments, the first supramolecular structures may be thermostable up to about 80° C., about 85° C., about 90° C., about 95° C., about 100° C., about 105° C., about 110° C., about 115° C., about 120° C., or about 125° C. In some embodiments, the supramolecular structures may be thermostable at between about 80° C. and about 125° C., between about 85° C. and about 120° C., between about 90° C. and 115° C., between about 95° C. and about 110° C., or between about 100° C. and about 105° C. The monoclonal clusters of a target oligonucleotide may be formed via solution-based capture using the described supramolecular structures. The monoclonal clusters of a target oligonucleotide may be generated by performing a local amplification of the target oligonucleotide captured by suitable chemically reactive groups bound directly or indirectly to the first supramolecular structures. In some embodiments, the target oligonucleotide is linked to a second nucleic acid supramolecular structure. In some embodiments, the second supramolecular structure is configured to release the target oligonucleotide in absence of an analyte molecule.

In some embodiments, the first nucleic acid supramolecular structure comprises a first chemically reactive group. In some embodiments, the first nucleic acid supramolecular structure further comprises the first core structure and a second chemically reactive group. In some embodiments, the first nucleic acid supramolecular structure is configured to be sufficiently thermostable to support a nucleic acid amplification. In some embodiments, the first chemically reactive group has an affinity to the target oligonucleotide.

In some embodiments, the second nucleic acid supramolecular structure further comprises a second core structure and a capture molecule, wherein the capture molecule is linked to the second core structure at a third location. In some embodiments, the target oligonucleotide is linked to the second core structure at a fourth location, wherein the fourth location is spatially separate from the third location. In some embodiments, the target oligonucleotide is configured to be released from the second core structure by providing a trigger to cleave the link between the target oligonucleotide and the second core structure in absence of the analyte molecule. In some embodiments, the target oligonucleotide remains to be bound to the second core structure through binding to the analyte molecule after providing the trigger to cleave the link between the target oligonucleotide and the second core structure in presence of the analyte molecule, wherein the analyte molecule also binds to the capture molecule at the third location of the second core structure. In some embodiments, the analyte molecule may be detected based on whether copies of the target oligonucleotide are formed on the hydrogel particle.

In some embodiments, the analyte molecule comprises a protein, a peptide, a peptide fragment, a lipid, a DNA, a RNA, an organic molecule, an inorganic molecule, complexes thereof, or any combinations thereof. In some embodiments, the trigger comprises DNA, RNA, a peptide, a small organic molecule, or combinations thereof.

By binding the first supramolecular structures, or a hydrogel matrix coating around the first supramolecular structures to a substrate, the monoclonal clusters may then be immobilized on the substrate. The substrate, in one embodiment, may be a single-molecule array having binding molecules, which may be used in downstream processing steps or operations, such as sequencing operations. In some embodiments, the single-molecule array comprises a nucleic acid supramolecular structure. In some embodiments, each nucleic supramolecular structure comprises corresponding binding molecules.

In some embodiments, the method comprises providing the target oligonucleotide and the hydrogel particle, incubating the hydrogel particle and the target oligonucleotide over a time period sufficient to facilitate capture of the target oligonucleotide by the first chemically reactive group, and performing a nucleic acid amplification of the captured target oligonucleotide using the nucleic acid primers thereby producing copies of the target oligonucleotide attached to the hydrogel particle. In some embodiments, the hydrogel particle comprises a nucleic acid supramolecular structure comprising a first chemically reactive group, a hydrogel matrix disposed around the nucleic acid supramolecular structure, and nucleic acid primers. In some embodiments, the nucleic acid supramolecular structure is configured to be sufficiently thermostable to support a nucleic acid amplification. In some embodiments, the first supramolecular structures may be thermostable up to about 80° C., about 85° C., about 90° C., about 95° C., about 100° C., about 105° C., about 110° C., about 115° C., about 120° C., or about 125° C. In some embodiments, the supramolecular structures may be thermostable at between about 80° C. and about 125° C., between about 85° C. and about 120° C., between about 90° C. and 115° C., between about 95° C. and about 110° C., or between about 100° C. and about 105° C. In some embodiments, the first chemically reactive group has an affinity for a target oligonucleotide. In some embodiments, the first nucleic acid supramolecular structure further comprises a first core structure and a second chemically reactive group linked to the first core structure at a second location. In some embodiments, the first chemically reactive group linked to the first core structure at a first location or a first set of locations.

In some embodiments, the nucleic acid primers are linked to the hydrogel matrix. In some embodiments, the nucleic acid primers facilitate amplification of the target oligonucleotide when the target oligonucleotide is captured by the first chemically reactive group. In some embodiments, the first chemically reactive group and the nucleic acid primers are extruded out of the hydrogel matrix. In some embodiments, the second location is spatially separated from the first location or the first set of locations. In some embodiments, the second chemically reactive group has an affinity to a corresponding binding molecule associated with a substrate.

### First Nucleic Acid Supramolecular Structure

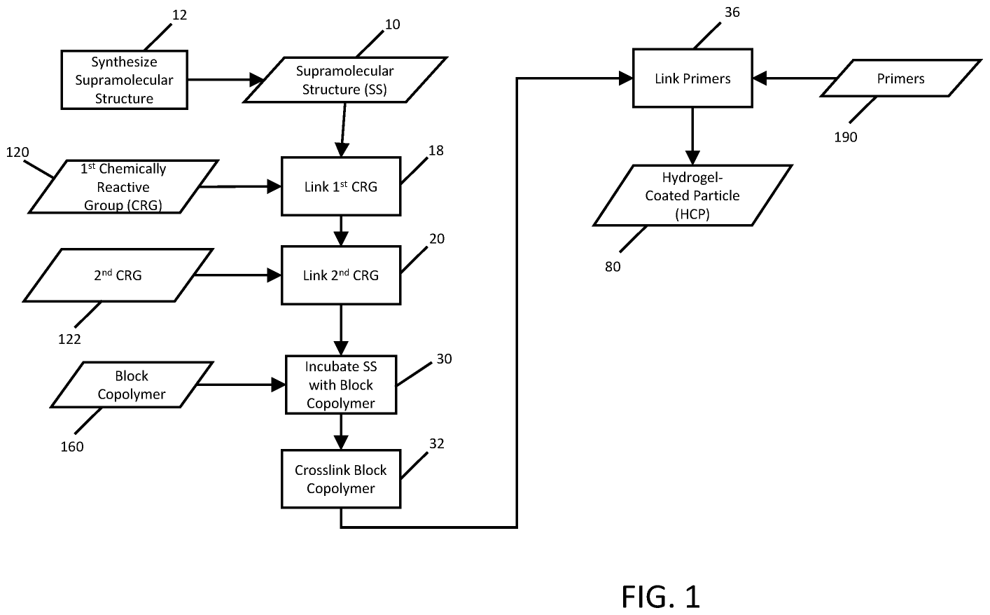

Accordingly, and with reference to the figures, various embodiments described herein utilize supramolecular structures 10. Turning to FIG. 1, such supramolecular structures 10 are acquired or synthesized (step 12) as part of forming a hydrogel-coated particle (HCP) 80 as described herein. In some embodiments, the supramolecular structure 10 comprises a core structure. In some embodiments, the core structure comprises one or more core molecules linked together. In some embodiments, the one or more core molecules comprise 2, 3, 4, 5, 6, 7, 8, 9, 10, 20, 50, 100, 200 or 500 unique molecules that are linked together. In some embodiments, the one or more core molecules comprises about 2 unique molecules to about 1000 unique molecules. In some embodiments, the one or more core molecules interact with each other and define the specific shape of the supramolecular structure. In some embodiments, the plurality of core molecules interacts with each other through reversible non-covalent interactions. In some embodiments, the specific shape of the core structure is a three-dimensional (3D) configuration. In some embodiments, the one or more core molecules provide a specific molecular weight. In some embodiments, the core structure 9 is a nanostructure. In some cases, the one or more core molecules comprise one or more nucleic acid strands (e.g., DNA, RNA, unnatural nucleic acids), one or more branched nucleic acids, one or more peptides, one or more small molecules, or combinations thereof. In some embodiments, the core structure 9 comprises a polynucleotide structure. In some embodiments, at least a portion of the core structure 9 is rigid. In some embodiments, at least a portion of the core structure 9 is semi-rigid. In some embodiments, at least a portion of the core structure 9 is flexible. In some embodiments, the core structure 9 comprises a scaffolded deoxyribonucleic acid (DNA) origami, a scaffolded ribonucleic acid (RNA) origami, a scaffolded hybrid DNA/RNA origami, a single-stranded DNA tile structure, a multi-stranded DNA tile structure, a single-stranded DNA origami, a single-stranded RNA origami, a single-stranded RNA tile structure, a multi-stranded RNA tile structures, a hierarchically composed DNA and/or RNA origami with multiple scaffolds, a peptide structure, or combinations thereof. In some embodiments, the DNA origami is scaffolded. In some embodiments, the RNA origami is scaffolded. In some embodiments, the hybrid DNA/RNA origami is scaffolded. In some embodiments, the core structure comprises a DNA origami, RNA origami, or hybrid DNA/RNA origami that comprises a prescribed two-dimensional (2D) or 3D shape. In some embodiments, the supramolecular structure 10 is a programmable structure that can spatially organize molecules. Further, in certain implementations the supramolecular structure 10 comprises a plurality of molecules linked together, some or all of which may interact with one another. The supramolecular structure 10 may have a specific shape or geometry, e.g., a substantially planar shape that has its longest dimension in an x-y plane. In some embodiments, the supramolecular structure 10 is a nanostructure, such as a nanostructure that comprises a prescribed molecular weight based on the plurality of molecules of the supramolecular structure 10. The plurality of molecules may, for example, be linked together through a bond, such as a chemical bond, a physical attachment, or combinations thereof. In certain implementations the supramolecular structure 10 comprises a large molecular entity, of specific shape and molecular weight, formed from a well-defined number of smaller molecules interacting specifically with each other. The structural, chemical, and physical properties of the supramolecular structure 10 may be explicitly designed. By way of example, the supramolecular structure 10 may comprise a plurality of subcomponents that are spaced apart according to a prescribed distance. In some embodiments, at least a portion of the supramolecular structure 10 (or its constituent core structure) is rigid or semi-rigid. Correspondingly or alternatively, all or parts of the supramolecular structure (or its constituent core structure) may be flexible or conformable. In certain embodiments the supramolecular structure 10 is at least 50 nm-200 nm in at least one dimension. In certain embodiments the supramolecular structure 10 is at least 20 nm long in any dimension.

In general, the supramolecular structure 10 may comprise a core structure which may be a polynucleotide structure, a protein structure, a polymer structure, or a combination thereof. In some embodiments, the core structure comprises one or more core molecules linked together. By way of example, the one or more core molecules may comprise 2, 3, 4, 5, 6, 7, 8, 9, 10, 20, 50, 100, 200 or 500 unique molecules that are linked together. In some embodiments, the one or more core molecules comprises about 2 unique molecules to about 1,000 unique molecules. In certain implementations, the one or more core molecules interact with each other and define the specific shape of the supramolecular structure 10. By way of example, the plurality of core molecules may interact with each other through reversible non-covalent interactions.

In some embodiments, the specific shape of the core structure of the supramolecular structure 10 has a three-dimensional (3D) configuration. Further, the one or more core molecules may provide a specific molecular weight. For example, all core structures of a plurality of supramolecular structures 10 may have the same configuration, size, and/or weight, but may differ in their attached linker sequences and/or other attached molecules, as described herein. However, excluding such differing linkers or other attached molecules, the supramolecular structures 10 of such a plurality may be otherwise identical. In certain examples the core structure may be a nanostructure. In some cases, the one or more core molecules comprise one or more nucleic acid strands (e.g., DNA, RNA, unnatural nucleic acids), one or more branched nucleic acids, one or more peptides, one or more small molecules, or combinations thereof. In some embodiments, the core structure 13 comprises an entirely polynucleotide structure.

In some embodiments, the supramolecular structure 10 (or is constituent core structure(s)) comprise a deoxyribonucleic acid (DNA) origami, a ribonucleic acid (RNA) origami, a hybrid DNA/RNA origami, a single-stranded DNA tile structure, a multi-strandedDNA tile structure, a single-stranded DNA origami, a single-stranded RNA origami, a single-stranded RNA tile structure, a multi-stranded RNA tile structures, a hierarchically composed DNA and/or RNA origami with multiple scaffolds, a peptide structure, an enzymatically synthesized nucleic acid structure (e.g., nanoball(s)), structures created by nucleic acid tile assembly, or combinations thereof. As discussed herein, in such embodiments the DNA origami, RNA origami, or hybrid DNA/RNA origami may be scaffolded. As used herein, the term “scaffold” or “scaffolded” refers to the use or inclusion of a circular ssDNA molecule, called a “scaffold” strand, that is folded into a predefined 2D or 3D shape by interacting with two or more short ssDNA, called “staple” strands, which interact with specific sub-sections of the ssDNA “scaffold” strand. In some embodiments, the core structure comprising a DNA origami, RNA origami, or hybrid DNA/RNA origami has a prescribed two-dimensional (2D) or 3D shape.

In an example embodiment, the core structure(s) of a supramolecular structure 10 may be a nucleic acid origami that comprises at least one lateral dimension between about 50 nm to about 1 μm. In an embodiment, the nucleic acid origami comprises at least one lateral dimension between about 50 nm to about 200 nm, about 50 nm to about 400 nm, about 50 nm to about 600 nm, about 50 nm to about 800 nm, about 100 nm to about 200 nm, about 100 nm to about 300 nm, about 100 nm to about 400 nm, about 100 nm to about 500 nm, or about 200 nm to about 400 nm by way of example. Further, in certain embodiments the nucleic acid origami comprises at least a first lateral dimension between about 50 nm to about 1 μm and a second lateral dimension, orthogonal to the first, between about 50 nm to about 1 μm. In one implementation the nucleic acid origami comprises a planar footprint having an area of about 200 nm² to about 1 μm².

In some embodiments, each component (e.g., constituent component) of the supramolecular structure 10 may be independently modified or tuned. By way of example, modifying one or more of the components of the supramolecular structure 10 may modify the 2D and 3D geometry of the supramolecular structure itself. In some embodiments, modifying one or more of the components of the supramolecular structure 10 may modify the 2D and 3D geometry of the core structure of the supramolecular structure 10. In some embodiments, such capability for independently modifying the components of the supramolecular nanostructure enables precise control over the organization of one or more supramolecular structures 10.

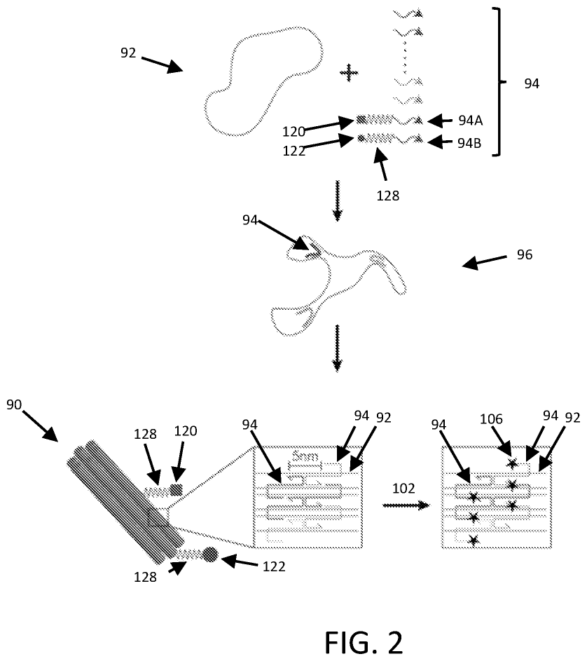

With the preceding high-level discussion of supramolecular structures 10, as used herein, in mind, and with reference to FIGS. 1 and 2, an example of a practical implementation of a synthesis step 12 of a suitable supramolecular structure 10 is provided. In this example, the synthesized supramolecular structure 10 is a scaffolded DNA origami 90. Turning to FIG. 2, in this example a scaffold 92 (e.g., a circular ssDNA molecule of known sequence, which may be referred to as a “scaffold” strand) may be combined with a plurality of staples 94 (e.g., two or more short ssDNA, called “staple” strands, which interact with specific sub-sections of the ssDNA “scaffold” strand). The staples 94 selectively bind to specified locations on the scaffold 92 such that a self-assembly (step 96) of the supramolecular structure 10, here a DNA origami 90 is performed. In particular, the self-assembly step 96 results in the scaffold 92 being folded into a predefined 2D or 3D shape via interactions with the staples 94. In one embodiment, the staples 94 are formed with excess thymine (T) located at nicks as well as crossovers so as to facilitate the crosslinking (i.e., formation of covalent crosslinks 106) of the staple structures 94 forming the DNA origami 90 when exposed to an energy source (e.g., UV illumination) at step 102. Such cross-linking may help improve the thermostability of the formed DNA origami. In practice, the cross-linking step 102 may be performed after the DNA origami 90 is purified away from any unattached staple strands 94.

In some embodiments, the nucleic acid supramolecular structure 10 comprises: a core structure, a first chemically reactive group linked to the core structure at a first location or a first set of locations. In some embodiments, the first chemically reactive group has an affinity to a specific oligonucleotide, and a second chemically reactive group linked to the core structure at a second location. In some embodiments, the second location is spatially separated from the first location or the first set of locations. In some embodiments, the second chemically reactive group has an affinity to a corresponding binding molecule associated with a substrate.

As also shown in FIG. 2, certain of the staples 94 may include attached chemically reactive groups, here denoted as a first chemically reactive group 120 and a second chemically reactive group 122. In this example, staples 94A and 94B are shown as respectively attached to the first chemically reactive group 120 and the second chemically reactive group 122. By way of further example, due to the selectivity of the staples 94 in terms of binding to specific locations on the scaffold 92, the first chemically reactive group 120 and the second chemically reactive group 122 may be selectively attached to respective staples 94 which selectively bind to scaffold 92 at spatially separated locations to ensure the separation of the first chemically reactive group 120 and the second chemically reactive group 122 on the DNA origami 90.

Turning back to FIG. 2, In the depicted example, the first chemically reactive group 120 and the second chemically reactive group 122 are respectively attached to staples 94A and 94B via polymer spacers 128 such that the first chemically reactive group 120 and the second chemically reactive group 122 are spatially separated from the DNA origami 90 by the length of the polymer spacers 128 when the DNA origami 90 (or other supramolecular structure 10) is formed, as shown in FIG. 2. In one embodiment, the polymer spacers 128 may be uncharged, flexible polymer spacers, such as polyethylene glycol (PEG).

In one embodiment the first chemically reactive group 120 and the second chemically reactive group 122 may be selected or configured so as to have minimal cross-reactivity. By way of non-limiting example, the first chemically reactive group 120 and the second chemically reactive group 122 may be any pair selected from the following list of reactive groups: a thiol group, an azide group, an amine group, or a carboxyl group.

Turning back to FIG. 1, it may be seen in this example that the linkage of the first chemically reactive group 120 and the second chemically reactive group 122 is depicted as separate and discrete steps 18 and 20 from the synthesis (step 12) of the supramolecular structure 10. However, as may be appreciated from the preceding example, in practice the linkage of the first and second chemically reactive groups 120, 122 may be contemporaneous with, and intrinsic to, the synthesis of the supramolecular structure 10, such an in the context of synthesizing a scaffolded supramolecular structure using staples as described herein. In some embodiments, the first chemically reactive group extends from the core structure by a polymer spacer.

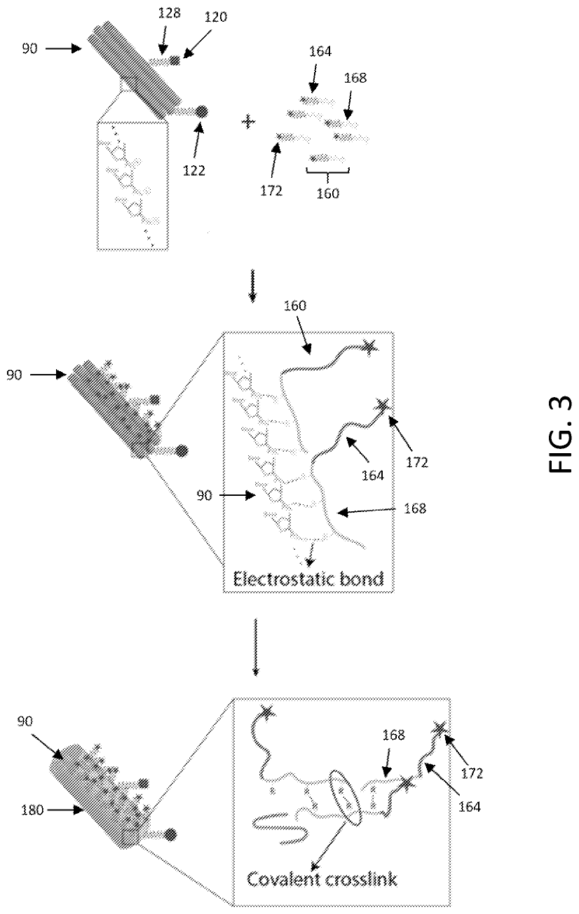

With reference to FIGS. 1 and 3, in certain embodiments the supramolecular structure 10 may be coated with a hydrogel to form a hydrogel-coated particle 80. By way of example, the purified supramolecular structure 10 (e.g., DNA origami 90) may be incubated (step 30, FIG. 1) with a block copolymer 160 (e.g., a diblock copolymer) having one or more charged regions, one or more uncharged regions, and a free terminal associated with an uncharged region and to which one or more reactive molecules are attached. By way of example, in one embodiment the block copolymer 160 is composed of one or more neutral polymers 164 (such as, but not limited to polyethylene glycol (PEG)), one or more charged polymers 168 (such as, but not limited to poly-L-lysine (PLL) or poly-acrylic acid (PAA)), and one or more reactive groups 172 (e.g., reactive molecules) on the PEG terminus.

As shown in FIG. 3, the electrostatic interaction between the charged section 168 of the block copolymers 160 and the DNA origami 90 leads to formation of a polyplex around the DNA origami 90. In circumstances in which the charged section 168 of the block copolymer 160 is positive, the precise cation concentration in solution is not important. If the charged section 168 of the block copolymer 160 is negative, the cations in solution should be either divalent or trivalent to enable formation of cationic sandwich between the

DNA origami 90 and the block copolymer 160. In some embodiments the thermostability of the polyplex may be improved by crosslinking (step 32, FIG. 1) the block copolymer 160 using an appropriate crosslinking agent (e.g., glutaraldehyde, bis(sulfosuccinimidyl)suberate (BS3), or di-tert-butyl peroxide (DTBP)) to yield a thin hydrogel matrix 180 around the DNA origami 90, as shown by the covalent crosslinks in FIG. 3. In some embodiments, the crosslinking agent comprises glutaraldehyde, bis(sulfosuccinimidyl)suberate (BS3), di-tert-butyl peroxide (DTBP), or combinations thereof.

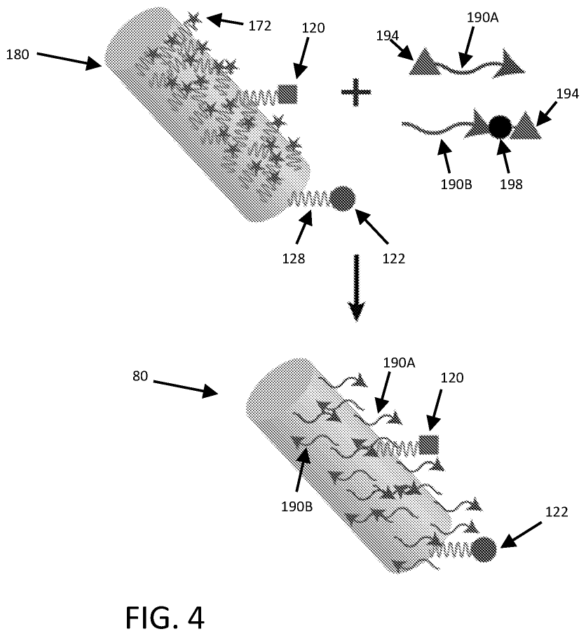

With reference to FIGS. 1 and 4, in certain implementations a pair of primers 190 are bound (step 36, FIG. 1) onto the reactive group 172 on the uncharged region terminus of the block copolymers 160 to yield a hydrogel-coated particle 80 having two reactive groups on its surface (i.e., the first chemically reactive group 120 and the second chemically reactive group 122) as well as a layer of primer pairs 190A and 190B. Binding of the primers 190 may be accomplished via complementarity of a complement molecule 194 (attached to the respective 3′ and 5′ ends of primers 190) with the reactive group 172. In the depicted example, one of the primers (primer 190B in this example) has a photocleavable linker 198, thereby allowing the primer 190B to be removed from the hydrogel-coated particle 80 upon irradiation with the appropriate light wavelength. In some embodiments, one primer of each pair of nucleic acid primers comprises a photocleavable linker so as to be removable upon irradiation by a suitable light source.

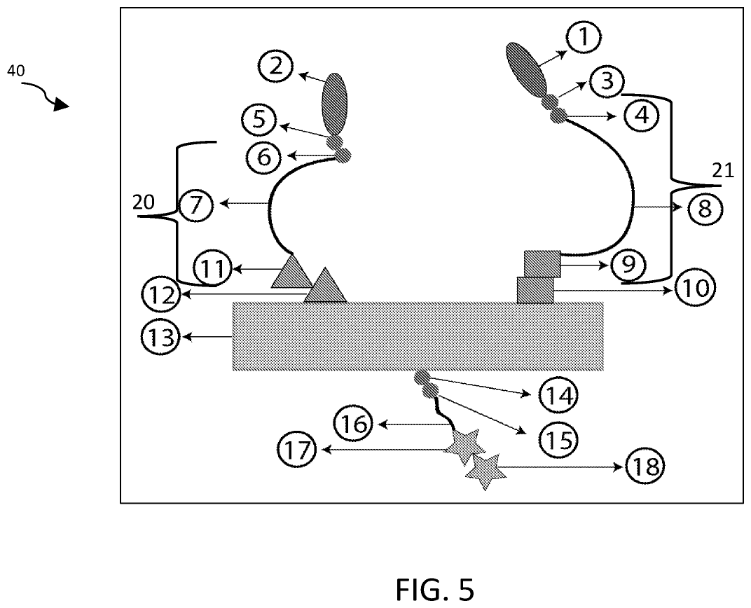

With the preceding in mind, the hydrogel-coated particle 80 as described above may be used to capture specific nucleic acid fragments (i.e., each hydrogel-coated particle 80 is specific to a specific nucleotide sequence, though different particles 80 may be specific to different sequences) in solution and to amplify captured fragments to form monoclonal clusters of the captured oligonucleotide(s) on the respective hydrogel-coated particles 80. In some embodiments, the specific nucleic acid fragments may be a target oligonucleotide. For example, turning to FIG. 5, in one implementation a hydrogel-coated particle 80 as described herein may be placed (step 220) in solution 228 in conjunction with a sample 224 containing oligonucleotide(s) (e.g., DNA fragments) of interest. It may be noted that in certain embodiments, a sample preparation step (e.g., a library preparation step) may be performed in which the oligonucleotides (e.g., DNA fragments) are ligated with an adaptor strand on either end of each fragment. In such a context, the adaptors may be composed of a primer binding region as well as a reactive group 200, ligated on one end of each fragment, which is complementary to the first chemically reactive group 120 of the hydrogel-coated particle 80. As may be appreciated, the reactive group 200 of the adaptor enables covalent bonding of the respective fragment (i.e., oligonucleotide) to the first chemically reactive group 120 on the hydrogel-coated particle 80. In some embodiments, a first adaptor strand and a second adaptor strand are independently ligated to each end of the target oligonucleotide, wherein the first adaptor strand comprises sequence complementary to one nucleic acid primer of the pair of nucleic acid primer and the second adaptor strand comprises sequence complementary to the other nucleic acid primer of the pair of nucleic acid primer. In some embodiments, the first adaptor strand comprises a reactive group, wherein the reactive group is configured to bind to the first chemically reactive group.

As used herein, the term “ligated” refers to the joining of two DNA fragments through the formation of a phosphodiester bond. An enzyme known as a DNA ligase catalyzes the formation of two covalent phosphodiester bonds between the 3′ hydroxyl group of one nucleotides and the 5′ phosphate group of another in an ATP dependent reaction.

The solution 228 containing the sample 224 having the prepared oligonucleotides and the hydrogel-coated particles 80 may be incubated, such as at 20° to 50° C. for a length of time ranging from a minute to an hour (step 234). In one implementation the oligonucleotides are incubated with the hydrogel-coated particles 80 in a denaturing condition (e.g., elevated temperature, in pure water, with 7M urea, and the like). The denaturing conditions limit or minimize interaction between the primer binding regions (ligated to the oligonucleotides in the sample preparation stage) and the primers 190 attached to the hydrogel-coated particles 80. The denaturing conditions during incubation, however, do not prevent interactions (e.g., binding) between the reactive group 200 ligated on one end of each oligonucleotide during sample preparation and the complementary first chemically reactive group 120 on the hydrogel-coated particle 80. In some embodiments, incubating the hydrogel particle and the target oligonucleotide over a time period is performed in a denaturing condition to avoid interaction between the adaptors and the nucleic acid primers.

Once the oligonucleotides are tethered to the hydrogel particles 80 by complementary pairing between the reactive group 200 of the adaptors and the first chemically reactive group 120 on the hydrogel-coated particle 80, excess untethered oligonucleotides may be filtered out and buffer exchanged for a non-denaturing buffer (i.e., the denaturing condition(s) are removed. In the absence of denaturing conditions the primer binding regions ligated to the oligonucleotides in the sample preparation stage and the primers 190 attached to the hydrogel-coated particles 80 may undergo complementary pairing. A local amplification step may then be performed (step 240). In the depicted example, enzymes 244 are added to the solution to facilitate the local amplification step. By way of example, one or more polymerases (e.g., Taq polymerase or a suitable variant) and deoxynucleoside triphosphate (dNTP) may be added to the solution. One or more rounds of thermocycling may then be performed to amplify the captured oligonucleotides onto the primers 190 attached to the hydrogel-coated particle(s) 80. The concentrations of the various solution constituents and the thermocycling conditions may be controlled so as to minimize or otherwise limit the interactions between hydrogel-coated particles 80. Alternatively, instead of employing thermocycling for polymerase chain reaction, local amplification of the captured oligonucleotides can instead be achieved isothermally using recombinase polymerase reaction. Once amplification is complete, each hydrogel-coated particle 80 is associated with a monoclonal cluster 250 comprised of copies of all or part of the oligonucleotide initially captured by the respective hydrogel-coated particle 80.

In some embodiments, each component of the supramolecular structure may be independently modified or tuned. In some embodiments, modifying one or more of the components of the supramolecular structure may modify the 2D and 3D geometry of the supramolecular structure itself. In some embodiments, modifying one or more of the components of the supramolecular structure may modify the 2D and 3D geometry of the core structure. In some embodiments, such capability for independently modifying the components of the supramolecular nanostructure enables precise control over the organization of one or more supramolecular structures on solid surfaces (e.g., planar surfaces or microparticles) and 3D volumes (e.g., within a hydrogel matrix).

### Second Nucleic Acid Supramolecular Structure

FIG. 5 provides an exemplary embodiment of the second nucleic acid supramolecular structure 40 comprising a second core structure 13, a capture molecule 2, a detector molecule 1, and an anchor molecule 18. In some embodiments, the second nucleic acid supramolecular structure comprises one or more capture molecules 2, and one or more detector molecules 1 and optionally one or more anchor molecules 18. In some embodiments, the second nucleic acid supramolecular structure does not comprise an anchor molecule. In some embodiments, the second nucleic acid supramolecular structure is a polynucleotide structure.

In some embodiments, the second core structure 13 comprises one or more core molecules linked together. In some embodiments, the one or more core molecules comprise 2, 3, 4, 5, 6, 7, 8, 9, 10, 20, 50, 100, 200 or 500 unique molecules that are linked together. In some embodiments, the one or more core molecules comprises about 2 unique molecules to about 1000 unique molecules. In some embodiments, the one or more core molecules interact with each other and define the specific shape of the supramolecular structure. In some embodiments, the plurality of core molecules interacts with each other through reversible non-covalent interactions. In some embodiments, the specific shape of the second core structure is a three-dimensional (3D) configuration. In some embodiments, the one or more core molecules provide a specific molecular weight. In some embodiments, the second core structure 13 is a nanostructure. In some cases, the one or more core molecules comprise one or more nucleic acid strands (e.g., DNA, RNA, unnatural nucleic acids), one or more branched nucleic acids, one or more peptides, one or more small molecules, or combinations thereof. In some embodiments, the one or more nucleic acid strands comprise a single stranded scaffold strand and more than two staple strands. In some embodiments, the second core structure comprises a polynucleotide structure. In some embodiments, at least a portion of the second core structure is rigid. In some embodiments, at least a portion of the second core structure is semi-rigid. In some embodiments, at least a portion of the second core structure is flexible. In some embodiments, the second core structure comprises a scaffolded deoxyribonucleic acid (DNA) origami, a scaffolded ribonucleic acid (RNA) origami, a scaffolded hybrid DNA/RNA origami, a single-stranded DNA tile structure, a multi-stranded DNA tile structure, a single-stranded DNA origami, a single-stranded RNA origami, a single-stranded RNA tile structure, a multi-stranded RNA tile structures, a hierarchically composed DNA and/or RNA origami with multiple scaffolds, a peptide structure, or combinations thereof. In some embodiments, the DNA origami is scaffolded. In some embodiments, the RNA origami is scaffolded. In some embodiments, the hybrid DNA/RNA origami is scaffolded. In some embodiments, the core structure comprising a DNA origami, RNA origami, or hybrid DNA/RNA origami that comprises a prescribed two-dimensional (2D) or 3D shape.

As shown in FIG. 5, in some embodiments, the second core structure 13 is configured to be linked to a capture molecule 2, a detector molecule 1, an anchor molecule 18, or combinations thereof. In some embodiments, the capture molecule 2, detector molecule 1, and/or anchor molecule 18 are immobilized with respect to the second core nanostructure 13 when linked thereto. In some embodiments, any number of the one or more core molecules comprises one or more core linkers 10, 12, 14 configured to form a linkage with a capture molecule 2, a detector molecule 1, and/or an anchor molecule 18. In some embodiments, any number of the one or more core molecules are configured to be linked with one or more core linkers 10, 12, 14 that are configured to form a linkage with a capture molecule 2, a detector molecule 1, and/or an anchor molecule 18. In some embodiments, one or more core linkers are linked to one or more core molecules through a chemical bond. In some embodiments, at least one of the one or more core linkers comprises a core reactive molecule. In some embodiments, each core reactive molecule independently comprises an amine, a thiol, a DBCO, an NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, at least one of the one or more core linkers comprises a DNA sequence domain. In some embodiments, one or more core linkers comprise at least one extended staple strands which particularly protrude from the second core structure. In some embodiments, the extended staple strands can be conjugated with (make a chemical bond with) a core reactive molecule. In some embodiments, the location of the extended staple strand is pre-determined.

In some embodiments, the second core structure 13 is linked to 1) a capture molecule 2 at a prescribed third location on the second core structure, 2) a detector molecule 1 at a prescribed fourth location on the second core structure, and optionally 3) an anchor molecule 18 at a prescribed fifth location on the second core structure. In some embodiments, a specified first core linker 12 is disposed at the third location on the second core structure, and a specified second core linker 10 is disposed at the fourth location on the second core structure. In some embodiments, one or more core molecules at the third location are modified to form a linkage with the first core linker 12. In some embodiments, the first core linker 12 is an extension of the core structure 13. In some embodiments, the first core linker 12 is an extended staple strand which particularly protrude from the second core structure 13. In some embodiments, one or more core molecules at the fourth location is modified to form a linkage with the second core linker 10. In some embodiments, the second core linker 10 is an extension of the second core structure 13. In some embodiments, the second core linker 10 is an extended staple strand which particularly protrude from the second core structure 13. In some embodiments, the 3D shape of the second core structure 13 and relative distances of the third and fourth locations are specified to maximize the intramolecular interactions between the capture molecule 2 and detector molecule 1. In some embodiments, the 3D shape of the second core structure 13 and relative distances of the third and fourth locations are specified to obtain a desired distance between the capture molecule 2 and detector molecule 1, so as to maximize the intramolecular interactions between the capture molecule 2 and detector molecule 1.

As described herein, in some embodiments, the distance between the capture molecule 2 and detector molecule 1 is about 3 nm, 4 nm, 5 nm, 6 nm, 10 nm, 12 nm, 15 nm, 20 nm, 30 nm, or 40 nm. In some embodiments, the distance between the capture molecule 2 and detector molecule 1 is about 1 nm to about 60 nm. In some embodiments, the distance between the capture molecule 2 and detector molecule 1 is about 1 nm to about 2 nm, about 1 nm to about 5 nm, about 1 nm to about 10 nm, about 1 nm to about 20 nm, about 1 nm to about 40 nm, about 1 nm to about 60 nm, about 2 nm to about 5 nm, about 2 nm to about 10 nm, about 2 nm to about 20 nm, about 2 nm to about 40 nm, about 2 nm to about 60 nm, about 5 nm to about 10 nm, about 5 nm to about 20 nm, about 5 nm to about 40 nm, about 5 nm to about 60 nm, about 10 nm to about 20 nm, about 10 nm to about 40 nm, about 10 nm to about 60 nm, about 20 nm to about 40 nm, about 20 nm to about 60 nm, or about 40 nm to about 60 nm, including increments therein. In some embodiments, the distance between the capture molecule 2 and detector molecule 1 is about 1 nm, about 2 nm, about 5 nm, about 10 nm, about 20 nm, about 40 nm, or about 60 nm. In some embodiments, the distance between the capture molecule 2 and detector molecule 1 is at least about 1 nm, about 2 nm, about 5 nm, about 10 nm, about 20 nm, or about 40 nm. In some embodiments, the distance between the capture molecule 2 and detector molecule 1 is at most about 2 nm, about 5 nm, about 10 nm, about 20 nm, about 40 nm, or about 60 nm.

As described herein, in some embodiments, the distance between the third location (corresponding to the capture molecule 2) and the fourth location (corresponding to the detector molecule 1) is about 3 nm, 4 nm, 5 nm, 6 nm, 10 nm, 12 nm, 15 nm, 20 nm, 30 nm, or 40 nm. In some embodiments, the distance between the capture molecule 2 and detector molecule 1 is about 1 nm to about 60 nm. In some embodiments, the distance the third location (corresponding to the capture molecule 2) and the fourth location (corresponding to the detector molecule 1) is about 1 nm to about 2 nm, about 1 nm to about 5 nm, about 1 nm to about 10 nm, about 1 nm to about 20 nm, about 1 nm to about 40 nm, about 1 nm to about 60 nm, about 2 nm to about 5 nm, about 2 nm to about 10 nm, about 2 nm to about 20 nm, about 2 nm to about 40 nm, about 2 nm to about 60 nm, about 5 nm to about 10 nm, about 5 nm to about 20 nm, about 5 nm to about 40 nm, about 5 nm to about 60 nm, about 10 nm to about 20 nm, about 10 nm to about 40 nm, about 10 nm to about 60 nm, about 20 nm to about 40 nm, about 20 nm to about 60 nm, or about 40 nm to about 60 nm, including increments therein. In some embodiments, the distance between the third location (corresponding to the capture molecule 2) and the fourth location (corresponding to the detector molecule 1) is about 1 nm, about 2 nm, about 5 nm, about 10 nm, about 20 nm, about 40 nm, or about 60 nm. In some embodiments, the distance between the capture molecule 2 and detector molecule 1 is at least about 1 nm, about 2 nm, about 5 nm, about 10 nm, about 20 nm, or about 40 nm. In some embodiments, the distance between the capture molecule 2 and detector molecule 1 the third location (corresponding to the capture molecule 2) and the fourth location (corresponding to the detector molecule 1) is at most about 2 nm, about 5 nm, about 10 nm, about 20 nm, about 40 nm, or about 60 nm.

In some embodiments, a specified third core linker 14 is disposed at the fifth location on the second core structure 13. In some embodiments, one or more core molecules at the fifth location is modified to form a linkage with the third core linker 14. In some embodiments, the third core linker 14 is an extension of the second core structure 13. In some embodiments, the third and second locations are disposed on a first side of the second core structure 13, and the optional third location is disposed on a second side of the second core structure 13. In some embodiments, the third core linker 14 comprises at least one extended staple strands which particularly protrude from the second core structure 13. In some embodiments, the extended staple strands can be conjugated with (make a chemical bond with) a core reactive molecule. In some embodiments, the location of the extended staple strand is pre-determined.

As shown in FIG. 5, in some embodiments, the anchor molecule 18 is linked to the core structure 13 through an anchor barcode. In some embodiments, the anchor barcode forms a linkage with the anchor molecule 18, and the anchor barcode forms a linkage with the core structure 13. In some embodiments, the anchor barcode comprises a first anchor linker 15, a second anchor linker 17, and an anchor bridge 16. In some embodiments, the first anchor linker 15 comprises a reactive molecule. In some embodiments, the first anchor linker 15 comprises a reactive molecule comprising an amine, a thiol, a DBCO, an NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the first anchor linker 15 comprises a DNA sequence domain. In some embodiments, the DNA sequence domain is complementary to DNA sequence domain of the third core linker 14. In some embodiments, the second anchor linker 17 comprises a reactive molecule. In some embodiments, the second anchor linker 17 comprises a reactive molecule comprising an amine, a thiol, a DBCO, an NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second anchor linker 17 comprises a DNA sequence domain. In some embodiments, the anchor bridge 16 comprises a polymer. In some embodiments, the anchor bridge 16 comprises a polymer that comprises a nucleic acid (DNA or RNA) of a specific sequence. In some embodiments, the anchor bridge 16 comprises a polymer such as PEG. In some embodiments, the first anchor linker 15 is attached to the anchor bridge 16 at a first terminal end thereof, and the second anchor linker 17 is attached to the anchor bridge 16 at a second terminal end thereof. In some embodiments, the first anchor linker 15 is attached to the anchor bridge 16 via a chemical bond. In some embodiments, the second anchor linker 17 is attached to the anchor bridge 16 via a physical attachment. In some embodiments, the first anchor linker 15 is attached to the anchor bridge 16 via a chemical bond. In some embodiments, the second anchor linker 17 is attached to the anchor bridge 16 via a physical attachment.

### Target Oligonucleotide

As shown in FIG. 5, in some embodiments, the detector molecule 1 is linked to the second core structure 13 through a target composition 21. In some embodiments, the target composition 21 forms a linkage with the detector molecule 1, and the target composition 21 forms a linkage with the second core structure 13. In some embodiments, the target composition 21 comprises a first detector linker 9, a second detector linker 4, and a target oligonucleotide 8. In some embodiments, the target composition 21 comprises the target oligonucleotide. In some embodiments, the target composition 21 may be the target oligonucleotide. In some embodiments, the first detector linker 9 comprises a reactive molecule. In some embodiments, the first detector linker 9 comprises a reactive molecule comprising an amine, a thiol, a DBCO, an NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the first detector linker 9 comprises a DNA sequence domain. In some embodiments, the DNA sequence domain is complementary to DNA sequence domain of the second core linker 10. In some embodiments, the second detector linker 4 comprises a reactive molecule. In some embodiments, the second detector linker 4 comprises a reactive molecule comprising an amine, a thiol, a DBCO, an NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the second detector linker 4 comprises a DNA sequence domain. In some embodiments, the DNA sequence domain is complementary to DNA sequence domain of a third detector linker 3. In some embodiments, the target composition comprises a target oligonucleotide 8. In some embodiments, the target oligonucleotide 8 comprises a DNA sequence domain. In some embodiments, the target oligonucleotide 8 comprises a polymer that comprises a nucleic acid (DNA or RNA) of a specific sequence. In some embodiments, the first detector linker 9 is attached to the target oligonucleotide 8 at a first terminal end thereof, and the second detector linker 4 is attached to the target oligonucleotide 8 at a second terminal end thereof. In some embodiments, the first detector linker 9 is attached to the target oligonucleotide 8 via a chemical bond. In some embodiments, the second detector linker 4 is attached to the target oligonucleotide 8 via a chemical bond. In some embodiments, the first detector linker 9 is attached to the target oligonucleotide 8 via a physical attachment. In some embodiments, the second detector linker 4 is attached to the target oligonucleotide 8 via a physical attachment.

In some embodiments, the target composition 21 is linked to the second core structure 13 through a linkage between the first detector linker 9 and the second core linker 10. In some embodiments, as described herein, the second core linker 10 is disposed at a fourth location on the second core structure 13. In some embodiments, the first detector linker 9 and second core linker 10 are linked together through a chemical bond. In some embodiments, the first detector linker 9 and second core linker 10 are linked together through a covalent bond. In some embodiments, the first detector linker 9 and second core linker 10 are linked together through hybridization between single stranded nucleic acids. In some embodiments, the linkage between the first detector linker 9 and second core linker 10 is reversible upon being subjected to a trigger. In some embodiments, the trigger comprises interaction with a deconstructor molecule or exposure to a trigger signal. In some embodiments, the detector deconstructor molecule comprises a nucleic acid (DNA or RNA), a peptide, a small organic molecule, or combinations thereof. In some embodiments the trigger signal comprises an optical signal. In some embodiments, the trigger signal comprises an electrical signal, microwave signal, ultraviolet illumination, visible illumination or near infra-red illumination.

In some embodiments, the target composition 21 is linked to the detector molecule 1 through a linkage between the second detector linker 4 and a third detector linker 3 bound to the detector molecule 1. In some embodiments, the third detector linker 3 comprises a reactive molecule. In some embodiments, the third detector linker 3 comprises a reactive molecule comprising an amine, a thiol, a DBCO, an NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the third detector linker 3 comprises a DNA sequence domain. In some embodiments, the specific DNA sequence domains of the third detector linker 3 and the second detector linker 4 are complementary to each other. In some embodiments, the detector molecule 1 is bound to the third detector linker 3 through a chemical bond. In some embodiments, the detector molecule 1 is bound to the third detector linker 3 through a covalent bond. In some embodiments, the second detector linker 4 and third detector linker 3 are linked together through a chemical bond. In some embodiments, the second detector linker 4 and third detector linker 3 are linked together through a covalent bond. In some embodiments, the third detector linker 3 and the second detector linker 4 are linked together through hybridization between single stranded nucleic acids. In some embodiments, the linkage between the second detector linker 4 and third detector linker 3 is reversible upon being subjected to a trigger. In some embodiments, the trigger comprises interaction with a deconstructor molecule or exposure to a trigger signal. In some embodiments, the target component 21 is configured to release the target oligonucleotide 8 from the second core structure 13.

In some embodiments, the second supramolecular structure comprises one or more stable state configurations. In some embodiments, the second supramolecular structure comprises one or more unstable state configurations. In some embodiments, the second supramolecular structure comprises a bi-stable configuration having a stable state configuration and an unstable state configuration. In some embodiments, the two states, stable and unstable are defined based on the ability of an individual supramolecular structure to remain structurally intact when subjected to a unique molecule (e.g., a deconstructor molecule) and/or a trigger signal. In some embodiments, when the second supramolecular structure is in the stable state, then all the different components that are part of the second supramolecular structure remain physically connected to each other even after being exposed to the deconstructor molecule and/or trigger signal. In some embodiments, when the second supramolecular structure is in the unstable state, then the exposure to the deconstructor molecule and/or trigger signal leads to a defined section (e.g., one or more subcomponents including the target oligonucleotide) of the supramolecular structure being physically cleaved, i.e. unbound (separated) from the supramolecular structure. In some embodiments, when the second supramolecular structure is in the unstable state, then the exposure to the deconstructor molecule and/or trigger signal leads to the target oligonucleotide being physically cleaved, i.e. unbound (separated) from the second supramolecular structure. In some embodiments, the second supramolecular structure is configured to shift from a stable state to an unstable state upon interaction with an analyte molecule (as described herein). In some embodiments, the second supramolecular structure is configured to shift from an unstable state to a stable state upon interaction with an analyte molecule (as described herein). In some embodiment, the analyte molecule that triggers the state change of the second supramolecular structure comprises a protein, clusters of proteins, peptide fragments, cluster of peptide fragments, DNA, RNA, DNA nanostructure, RNA nanostructures, lipids, an organic molecule, an inorganic molecule, or any combination thereof.

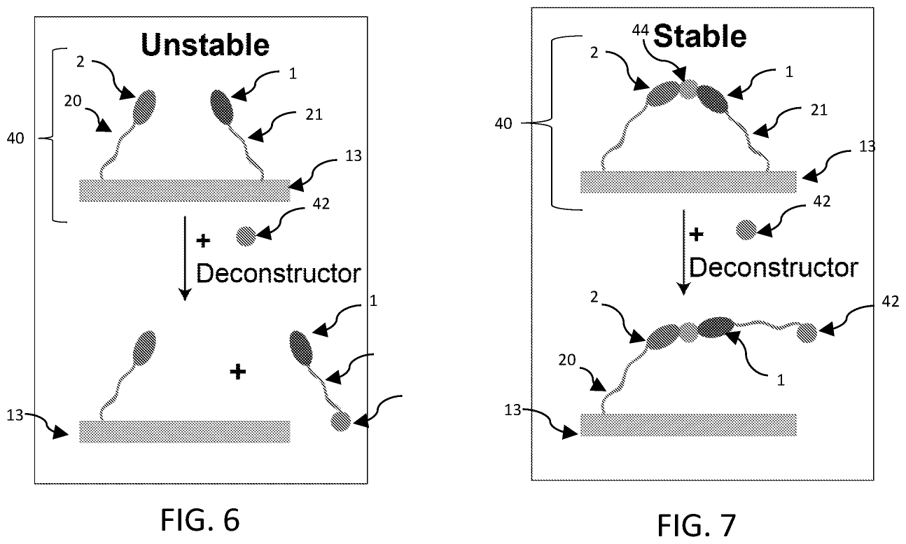

In some embodiments, the second supramolecular structure in an unstable state configuration comprises a physical state wherein a linkage between the core structure 13 and a capture molecule 2 may be cleaved such that the capture molecule 2 is unbound from the second core nanostructure 13. In some embodiments, the unstable state configuration comprises a physical state wherein a linkage between the second core nanostructure 13 and a detector molecule 1 may be cleaved such that the detector molecule 1 is unbound from the core nanostructure 13. In some embodiments, the unstable state configuration comprises a physical state wherein a linkage between the second core nanostructure 13 and a capture molecule 2 and a linkage between the second core nanostructure 13 and a detector molecule 1 may be cleaved such that the capture molecule 2 and detector molecule 1 are unbound from the second core nanostructure 13. In some embodiments, the linkage between the second core nanostructure 13 and 1) the capture molecule 2, 2) the detector molecule 1, or 3) both, are cleaved upon being subjected to a trigger (e.g., a deconstructor molecule as described herein or trigger signal as described herein). FIG. 6 provides an exemplary depiction of a supramolecular structure 40 in an unstable state, wherein the detector molecule 1 is initially linked to the target component 21 comprising the target oligonucleotide 8 and also is bound to the second core structure 13. With continued reference to FIG. 6, interaction with a deconstructor molecule 42 subsequently cleaves the linkage between the target component 21, comprising the target oligonucleotide 8, and the second core structure 13, such that the detector molecule 1 is unbound from the second core nanostructure 13. In some embodiments, in the unstable state, the capture molecule 2 and detector molecule 1 on the second core nanostructure 13 are freely diffusing with respect to each other, constrained only by the physical configuration of the second core nanostructure 13. In some embodiments, in the unstable state, the target component 21 comprising the target oligonucleotide 8 is freely diffusing from the second core nanostructure 13. In some embodiments, in unstable state, the target component 21, comprising the target oligonucleotide 8, is available to the binding with the first chemically reactive group of the first core structure.

In some embodiments, the stable state configuration comprises a physical state wherein the capture molecule 2 remains bound to the second core nanostructure 13 upon cleavage of a linkage between the second core structure 13 and the capture molecule 2. In some embodiments, the stable state configuration comprises a physical state wherein the detector molecule 1 remains bound to the second core structure 13 upon cleavage of a linkage between the second core nanostructure 13 and the detector molecule 1. In some embodiments, the stable state configuration comprises a physical state wherein the capture molecule 2 and detector molecule 1 are proximally positioned with respect to each other. In some embodiments, the detector molecule 1 and capture molecule 2 are proximally positioned with respect to each other with, or without, explicit bond formation between each other. In some embodiments, the detector 1 and capture 2 molecules are linked to each other. In some embodiments, the detector 1 and capture 2 molecules are linked to each other through a chemical bond. In some embodiments, the detector 1 and capture 2 molecules are linked together through a linkage with another molecule located between the capture and detector molecules (e.g., a sandwich formation). In some embodiments, the detector and capture molecules are linked together through linkage with an analyte molecule 44 from a sample. FIG. 7 provides an exemplary depiction of a supramolecular structure 40 in a stable state, wherein the capture molecule 2 is linked to the detector molecule 1 through linkage with an analyte molecule 44. With continued reference to FIG. 7, interaction with a deconstructor molecule 42 cleaves the linkage between the detector molecule 1 and second core structure 13, but the detector molecule 1 remains bound to the second core nanostructure 13 through the linkage with the capture molecule 2. In some embodiments, the target component comprising the target oligonucleotide remains bound to the second core nanostructure 13 through the linkage with the capture molecule 2. As described further herein, in some embodiments, a capture and/or detector molecule is configured to form a linkage with one or more specific types of analyte molecule from the sample. In some embodiments, interaction with the deconstructor molecule and/or trigger signal does not cleave the linkage between the capture and detector molecules. In some embodiments, in the stable state, the target component 21, comprising the target oligonucleotide 8, is not released from the second core nanostructure 13. In some embodiments, in stable state, the target component 21, comprising the target oligonucleotide 8, is available to the binding with the first chemically reactive group of the first core structure.

In some embodiments, the capture molecule 2 comprises a protein, a peptide, an antibody, an aptamer (RNA and DNA), a fluorophore, a nanobody, a darpin, a catalyst, a polymerization initiator, a polymer like PEG, an organic molecule, or combinations thereof. In some embodiments, the detector molecule 1 comprises a protein, a peptide, an antibody, an aptamer (RNA and DNA), a fluorophore, a nanobody, a darpin, a catalyst, a polymerization initiator, a polymer like PEG, an organic molecule, or combinations thereof. In some embodiments, the anchor molecule comprises a reactive molecule. In some embodiments, the anchor molecule 18 comprises a reactive molecule. In some embodiments, the anchor molecule 18 comprises a DNA strand comprising a reactive molecule. In some embodiments, the anchor molecule 18 comprises an amine, a thiol, a DBCO, an NHS ester, a maleimide, biotin, an azide, an acrydite, a single stranded nucleic acid (e.g., RNA or DNA) of specific sequence, or a polymer (e.g., polyethylene glycol (PEG) or one or more polymerization initiators). In some embodiments, the anchor molecule 18 comprises a protein, a peptide, an antibody, an aptamer (RNA and DNA), a flourophore, a nanobody, a darpin, a catalyst, a polymerization initiator, a polymer like PEG, an organic molecule or combinations thereof. In some embodiments, a single pair of a capture molecule 2 and corresponding detector molecule 1 is linked to the second core structure 13. In some embodiments, a plurality of pairs of capture molecules 2 and corresponding detector molecules 1 are linked to a second core structure 13. In some embodiments, the plurality of pairs of capture molecules 2 and corresponding detector molecules 1 are spaced apart from each other to minimize crosstalk, i.e. minimizing capture and/or detector molecules from a first pair interacting with capture and/or detector molecules from a second pair.

### Formation of Monoclonal Clusters

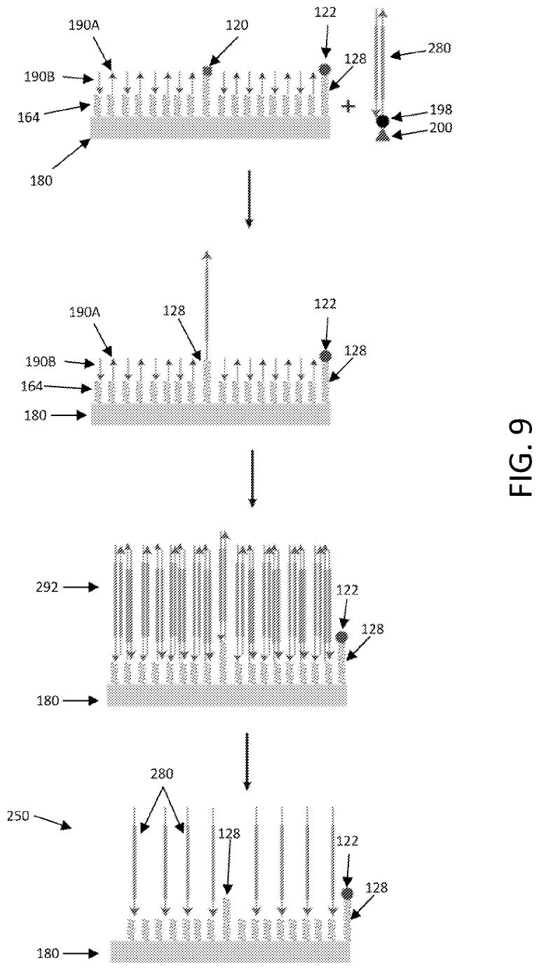

Turning to FIG. 9, aspects of this process are further illustrated using representational images. As shown in this figure, a hydrogel coating or surface 180 of the hydrogel-coated particle 80 is provided on which the first chemically reactive group 120 and the second chemically reactive group 122 (attached via polymer spacers 128) are provided. Also on the hydrogel coating or surface 180 are pairs of primers 190 (i.e., primers 190A and 190B) attached via neutral polymers 164.

In solution with the hydrogel-coated particles are oligonucleotides 280 (shown here initially in a double-stranded form) which have undergone sample preparation. In some embodiments, the oligonucleotide 280 comprises the target oligonucleotide. By way of example, an oligonucleotide strand 280 (e.g., an ssDNA fragment with a reactive group or molecule 200 on its 5′ terminus) is incubated in a denaturing environment with the hydrogel-coated particles 80. In certain embodiments, oligonucleotides 280 that have not been modified with adaptors may be purified from the solution. As shown in FIG. 9, in the denaturing environment, the modified oligonucleotide strand 280 having the reactive group 200 reacts with and/or otherwise binds to the first chemically reactive group 120 on the hydrogel-coated particle 80.

Subsequently the de-naturing buffer may be exchanged for a non-denaturing buffer (i.e., the denaturing condition(s) are removed, allowing primer binding regions on the oligonucleotides 280 to pair with the primers 190 attached to the hydrogel-coated particles 80. In some embodiments, prior to the nucleic acid amplification, exchanging the denaturing condition to a non-denaturing condition allows interaction between the adaptors and the nucleic acid primers. A bridge amplification step may then be performed by thermocycling in the presence of polymerase and dNTP so as to produce multiple copies 292 of the oligonucleotide 280. Alternatively, as noted above, an isothermal recombinase polymerase reaction process may be used for local amplification in place of a thermocycle-based polymerase chain reaction. The original attached oligonucleotide 280 may be photocleaved under denaturing conditions at the conclusion of the amplification process, resulting in amplified strands complementary to the attached oligonucleotide 280 being denatured and removed. As a result only copies of the oligonucleotide 280 are left attached to the hydrogel coating or surface 180 so as to form a monoclonal cluster 250.

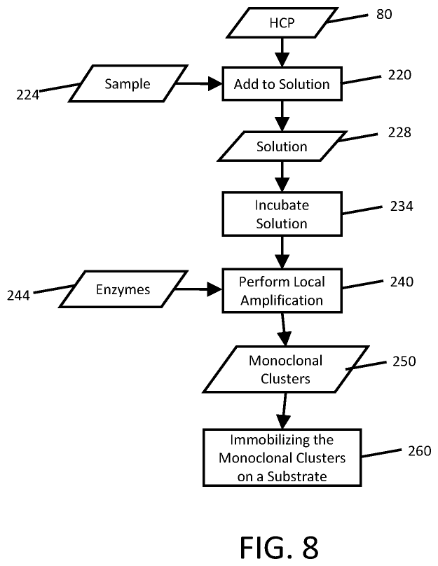

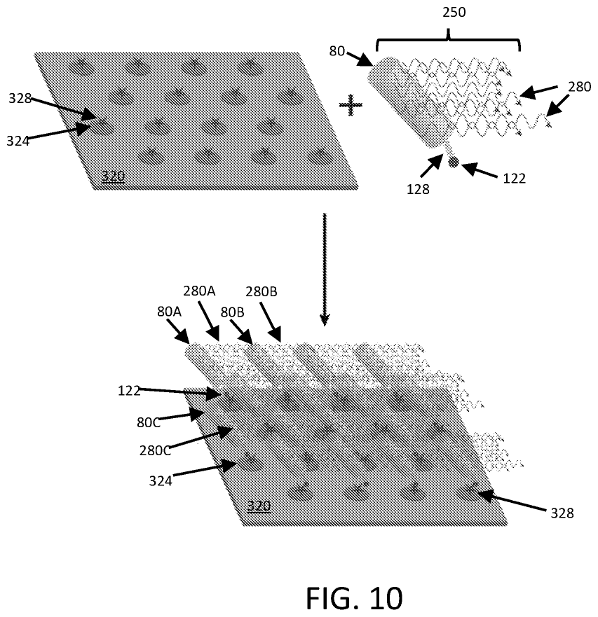

In accordance with further aspects of the present disclosure, the monoclonal clusters 250 created in this manner can subsequently be immobilized (step 260, FIG. 8) on a surface, such as a patterned surface, and further processed. By way of example, and turning to FIG. 10, in accordance with one implementation the monoclonal clusters 250 on the hydrogel-coated particles 80 may be organized on a surface and characterized, such as by a sequencing technique (e.g., sequence-by-synthesis (SBS)).

In the example shown in FIG, 10, the monoclonal clusters 250 formed on the hydrogel-coated particles 80 are organized on a planar surface or substrate 320 in a regular pattern. In the depicted example, the hydrogel-coated particles 80 (e.g., hydrogel-coated particles 80A, 80B, 80C), each coated with copies of different respective specific oligonucleotides 280 (e.g., oligonucleotides 280A, 280B, 280C), are incubated with a single molecule array of nucleic acid supramolecular structures 324, such as DNA origami, placed on the surface 320. By way of example, the single molecule array of nucleic acid supramolecular structures 324 (e.g., DNA origami) may be formed as a grid of binding sites on the surface 320 using DNA origami placement (DOP) techniques.

Each nucleic acid supramolecular structure 324 placed on the surface 320 carries a single capture molecule 328 capable of covalently interacting with (or otherwise attach to) the second chemically reactive group 122 on the hydrogel-coated particles 80. After the incubation period (which may be at 20° to 50° C. for a length of time ranging from a minute to three hours) unbound hydrogel-coated particles may be removed by performing one or more buffer washes. The binding site locations of the substrate 320, corresponding to the initial locations of nucleic acid supramolecular structures, will each have an individual monoclonal cluster (i.e., hydrogel coated particle 80 and associated oligonucleotide 280 copies) covalently immobilized and ready for subsequent processing (e.g., sequencing). In some embodiments, the hydrogel particle is immobilized on a substrate by a binding between the second chemically reactive group disposed on the hydrogel particle and a corresponding binding molecule attached to the substrate. In some embodiments, the second chemically reactive group comprises a thiol reactive group, an azide reactive group, an amine reactive group, or a carboxyl reactive group. In some embodiments, the substrate comprises a single-molecule array. In some embodiments, the single-molecule array comprises a second nucleic acid supramolecular structure, each second nucleic supramolecular structure comprising the corresponding binding molecule. In some embodiments, the second nucleic acid supramolecular structure comprises a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, an enzymatically synthesized nucleic acid structure, a nucleic acid structures created by tile assembly, or combinations thereof. In some embodiments, sequencing the target oligonucleotide may be performed after the nucleic acid amplification.

With the preceding in mind, it should be appreciated that there are variations to the techniques described above that are contemplated and within the scope of the present disclosure. For example, while the preceding discussion describes implementations in which the captured oligonucleotides 280 are amplified on the hydrogel-coated particles 80 in solution, i.e., prior to an immobilization step, in alternative approaches the amplification of the oligonucleotides 280 may be performed after the hydrogel-coated particle is immobilized on a patterned surface. In some embodiments, the captured oligonucleotide 280 comprises the target oligonucleotide. By way of example, in such an approach the hydrogel-coated particles 80 and covalently attached oligonucleotides 280 may be incubated on the single molecule array of capture sites prior to amplification, thereby immobilizing the hydrogel-coated particles with respect to the capture sites, created by DOP techniques, on the surface 320. The immobilized hydrogel-coated particles 80 and attached oligonucleotides may then be used in an amplification process by incubating the substrate 320 with a suitable polymerase and dNTP followed by thermocycling (e.g., 20-30 rounds of thermocycling). The resulting product should correspond to the affixed monoclonal clusters 250 on the substrate 320 as described above.

### Without Hydrogel Matrix Coating

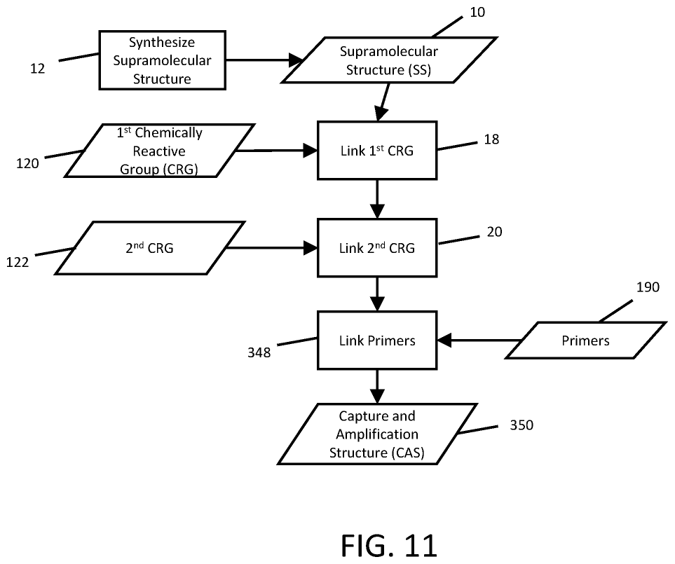

While the preceding describes approaches in which a hydrogel-coated supramolecular structure 10 is employed, it may also be appreciated that in other embodiments the hydrogel matrix coating the supramolecular structure 10 may be omitted. In such approaches the primers 190 may instead be linked to the supramolecular structure 10 via the staples 94 used in forming the supramolecular structure 10 as described with respect to the first chemically reactive group 120 and the second chemically reactive group 122. In such approaches, the primers 190 may be directly attached to the staples 94 itself as extensions or by having a particular reactive group on the staples themselves. Of note, this approach may limit or otherwise constrain the design space available since the sequence of the primer region needs to be designed to reduce the possibility of crosslinking within that region. Due to the absence of a hydrogel-coating, the particle formed in such an approach, e.g., a supramolecular structure 10 having a first chemically reactive group 120, a second chemically reactive group 122, and linked primers 190 may instead be characterized as a capture and amplification structure 350, as shown in FIG. 11.

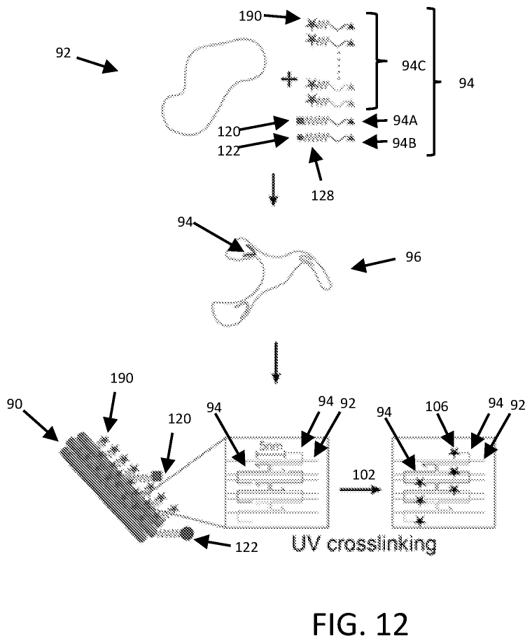

This approach is further illustrated in FIG. 12. In this example, the synthesized supramolecular structure 10 is a scaffolded DNA origami 90. In this example the scaffold 92 may be combined with a plurality of staples 94 which interact with specific sub-sections of the scaffold 92. The staples 94 selectively bind to specified locations on the scaffold 92 such that a self-assembly (step 96) of the supramolecular structure 10, here a DNA origami 90 is performed. In particular, the self-assembly step 96 results in the scaffold 92 being folded into a predefined 2D or 3D shape via interactions with the staples 94. In one embodiment, the staples 94 are formed with excess thymine (T) located at nicks as well as crossovers so as to facilitate the crosslinking (i.e., formation of covalent crosslinks 106) of the staple structures 94 forming the DNA origami 90 when exposed to an energy source (e.g., UV illumination) at step 102. Such cross-linking may help improve the thermostability of the formed DNA origami. In practice, the cross-linking step 102 may be performed after the DNA origami 90 is purified away from any unattached staple strands 94. In some embodiments, the nucleic acid supramolecular structure is configured to be thermostable via UV irradiation. In some embodiments, the hydrogel matrix comprises a thermostable hydrogel matrix.

As also shown in FIG. 12, certain of the staples 94 may include attached chemically reactive groups, here denoted as a first chemically reactive group 120, a second chemically reactive group 122, and primers (e.g., primer pairs) 190. In this example, staples 94A, 94B, and 94C are shown as respectively attached to the first chemically reactive group 120, the second chemically reactive group 122, and the primers 190. By way of further example, due to the selectivity of the staples 94 in terms of binding to specific locations on the scaffold 92, the first chemically reactive group 120, the second chemically reactive group 122, and the primers 190 may be selectively attached to respective staples 94 which selectively bind to scaffold 92 at spatially separated and specified locations on the DNA origami 90.

Turning back to FIG. 11, it may be seen in this example that the linkage of the first chemically reactive group 120, the second chemically reactive group 122, and the primers 190 is depicted as separate and discrete steps 18, 20, and 348 from the synthesis (step 12) of the supramolecular structure 10. However, as may be appreciated from the preceding example, in practice the linkage of the first and second chemically reactive groups 120, 122 and the primers 190 may be contemporaneous with, and intrinsic to, the synthesis of the supramolecular structure 10, such an in the context of synthesizing a scaffolded supramolecular structure using staples as described herein.

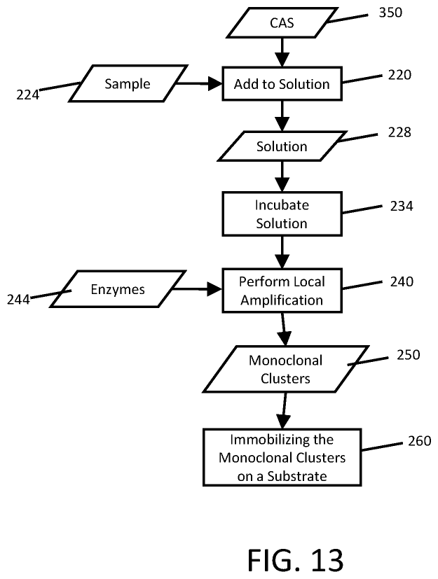

With the context of FIGS. 11 and 12 in mind, FIG. 13 illustrates a process flow corresponding to the process steps illustrated in FIG. 8, but in the context of utilizing a capture and amplification structure 350 which does not include a hydrogel coating.

Also provided herein in one embodiment is a method for producing a monoclonal cluster of a target oligonucleotides on a particle. The method comprises providing the target oligonucleotide and a capture and amplification structure, incubating the capture and amplification structure with a second nucleic acid supramolecular structure, comprising the target oligonucleotide, over a time period sufficient to facilitate capture the target oligonucleotide by the first chemically reactive group, and performing a nucleic acid amplification of the captured target oligonucleotide using the nucleic acid primers thereby producing copies of the target oligonucleotide attached to the capture and amplification structure. In some embodiments, the capture and amplification structure comprise a first nucleic acid supramolecular structure comprising a plurality of crosslink molecules and a first chemically reactive group. In some embodiments, the second supramolecular structure is configured to release the target oligonucleotide in absence of an analyte molecule. In some embodiments, the first chemically reactive group having an capture affinity for the target oligonucleotide. In some embodiments, nucleic acid primers are linked to the plurality of crosslink molecules. In some embodiments, the nucleic acid primers amplify or facilitate amplification of the specific target oligonucleotide when bound by the chemically reactive group.

In some embodiments, the first nucleic acid supramolecular structure further comprises a first core structure and a second chemically reactive group linked to the first core structure at a second location. In some embodiments, the first chemically reactive group linked to the first core structure at a first location or a first set of locations. In some embodiments, the second location is spatially separated from the first location or the first set of locations. In some embodiments, the second chemically reactive group has an affinity to a corresponding binding molecule associated with a substrate. In some embodiments, the first core structure comprises a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, an enzymatically synthesized nucleic acid structure, a nucleic acid structure created by tile assembly, or combinations thereof. In some embodiments, the first nucleic acid supramolecular structure is configured to be thermostable via UV irradiation. In some embodiments, the first supramolecular structures may be thermostable up to about 80° C., about 85° C., about 90° C., about 95° C., about 100° C., about 105° C., about 110° C., about 115° C., about 120° C., or about 125° C. In some embodiments, the supramolecular structures may be thermostable at between about 80° C. and about 125° C., between about 85° C. and about 120° C., between about 90° C. and 115° C., between about 95° C. and about 110° C., or between about 100° C. and about 105° C.

In some embodiments, the nucleic acid amplification comprises a local amplification. In some embodiments, a first adaptor strand and a second adaptor strand are independently ligated to each end of the target oligonucleotide, wherein the first adaptor strand comprises sequence complementary to one nucleic acid primer of the pair of nucleic acid primer and the second adaptor strand comprises sequence complementary to the other nucleic acid primer of the pair of nucleic acid primer. In some embodiments, the first adaptor strand comprises a reactive group, wherein the reactive group is configured to bind to the first chemically reactive group.

In some embodiments, incubating the hydrogel particle and the target oligonucleotide over a time period is performed in a denaturing condition to avoid interaction between the adaptors and the nucleic acid primers. In some embodiments, the denaturing condition comprises elevated temperature, water without any chemicals, 7 M urea, or combinations thereof. In some embodiments, prior to the nucleic acid amplification, exchanging the denaturing condition to a non-denaturing condition may allow interaction between the adaptors and the nucleic acid primers. In some embodiments, performing the nucleic acid amplification comprises adding enzymes. In some embodiments, performing the nucleic acid amplification comprises using a thermocycler.

In some embodiments, the particle is immobilized on a substrate. In some embodiments, the particle is immobilized on the substrate by a binding between the second chemically reactive group disposed on the particle and a corresponding binding molecule attached to the substrate. In some embodiments, the second chemically reactive group comprises a thiol reactive group, an azide reactive group, an amine reactive group, or a carboxyl reactive group. In some embodiments, the substrate comprises a single-molecule array. In some embodiments, the single-molecule array comprises a second nucleic acid supramolecular structure, each second nucleic supramolecular structure comprising the corresponding binding molecule. In some embodiments, the second nucleic acid supramolecular structure comprises a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, an enzymatically synthesized nucleic acid structure, a nucleic acid structures created by tile assembly, or combinations thereof. In some embodiments, sequencing of the target oligonucleotide may be performed after the nucleic acid amplification.

In some embodiments. the first chemically reactive group comprises a thiol reactive group, an azide reactive group, an amine reactive group, or a carboxyl reactive group. In some embodiments, unbound oligonucleotides may be filtered out prior to performing the nucleic acid amplification.

### Formation of a Monoclonal Cluster of a Target Oligonucleotide

Also provided herein is a method for forming a monoclonal cluster of a target oligonucleotide on a hydrogel particle. In some embodiments, the method comprises (i) incubating a first nucleic acid supramolecular structure, comprising a first chemically reactive group, with a plurality of polymer molecules to form a hydrogel matrix around the first nucleic acid supramolecular structure, (ii) providing a crosslinking agent to the hydrogel matrix around the first nucleic acid supramolecular structure to form the hydrogel particle, (iii) incubating the hydrogel particle with a second nucleic acid supramolecular structure, comprising the target oligonucleotide, sufficiently enough to facilitate capture of the target oligonucleotide by the first chemically reactive group, and (iv) amplifying the target oligonucleotide thereby producing copies of the target oligonucleotide attached to the hydrogel particle. In some embodiments, the first nucleic acid supramolecular structure is configured to be sufficiently thermostable to support a nucleic acid amplification. In some embodiments, the supramolecular structures may be thermostable up to about 80° C., about 85° C., about 90° C., about 95° C., about 100° C., about 105° C., about 110° C., about 115° C., about 120° C., or about 125° C. In some embodiments, the supramolecular structures may be thermostable at between about 80° C. and about 125° C., between about 85° C. and about 120° C., between about 90° C. and 115° C., between about 95° C. and about 110° C., or between about 100° C. and about 105° C. In some embodiments, the first chemically reactive group has an affinity to the target oligonucleotide. In some embodiments, the second supramolecular structure is configured to release the target oligonucleotide in absence of an analyte molecule.

In some embodiments, the first nucleic acid supramolecular structure further comprises the first core structure and a second chemically reactive group. In some embodiments, the first chemically reactive group is linked to the first core structure at a first location or a first set of locations. In some embodiments, the second chemically reactive group is linked to the first core structure at a second location, wherein the second location is spatially separated from the first location or the first set of locations. In some embodiments, the second chemically reactive group has an affinity to a corresponding binding molecule associated with a substrate. In some embodiments, the first location or the first set of locations and the second location are separated spatially enough to avoid cross-reactivity between the first chemically reactive group and the second chemically reactive group.

In some embodiments, the plurality of polymer molecules comprises a block copolymer comprising one or more charged regions, one or more uncharged regions, a free terminal associated with the uncharged region, and one or more reactive molecules attached to the free terminal. In some embodiments, the one or more uncharged regions comprises a neutral polymer. In some embodiments, the neutral polymer comprises polyethylene glycol (PEG). In some embodiments, the one or more charged region comprises a charged polymer. In some embodiments, the charged polymer comprises poly-L-lysine (PLL) or poly-acrylic acid (PAA).

In some embodiments, the second nucleic acid supramolecular structure further comprises a second core structure and a capture molecule, wherein the capture molecule is linked to the second core structure at a third location. In some embodiments, the target oligonucleotide is linked to the second core structure at a fourth location, wherein the fourth location is spatially separate from the third location. In some embodiments, the target oligonucleotide is configured to be released from the second core structure by providing a trigger to cleave the link between the target oligonucleotide and the second core structure in absence of the analyte molecule. In some embodiments, the target oligonucleotide remains to be bound to the second core structure through binding to the analyte molecule after providing the trigger to cleave the link between the target oligonucleotide and the second core structure in presence of the analyte molecule, wherein the analyte molecule also binds to the capture molecule at the third location of the second core structure.

In some embodiments, the analyte molecule may be detected based on whether copies of the target oligonucleotide are formed on the hydrogel particle. In some embodiments, the analyte molecule comprises a protein, a peptide, a peptide fragment, a lipid, a DNA, a RNA, an organic molecule, an inorganic molecule, complexes thereof, or any combinations thereof. In some embodiments, the trigger comprises DNA, RNA, a peptide, a small organic molecule, or combinations thereof. In some embodiments, the analyte molecule binds to the capture molecule through a chemical bond and/or binds to the target oligonucleotide through a chemical bond. In some embodiments, the capture molecule comprises a protein, a peptide, an antibody, an aptamer (RNA and DNA), a fluorophore, a darpin, a catalyst, a polymerization initiator, a polymer like PEG, or combinations thereof.

In some embodiments, the crosslinking agent comprises glutaraldehyde, bis(sulfosuccinimidyl)suberate (BS3), di-tert-butyl peroxide (DTBP), or combinations thereof. In some embodiments, nucleic acid primers are linked to the hydrogel matrix. In some embodiments, the nucleic acid primers facilitate amplification of the target oligonucleotide when the target oligonucleotide is captured by the first reactive group. In some embodiments, the first reactive group and the nucleic acid primers are extruded out of the hydrogel matrix. In some embodiments, the first nucleic acid supramolecular structure is configured to be thermostable via UV irradiation. In some embodiments, the first core structure and the second core structure independently comprise a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, an enzymatically synthesized nucleic acid structure, a nucleic acid structure created by tile assembly, or combinations thereof. In some embodiments, amplifying the target oligonucleotide comprises a local amplification. In some embodiments, amplifying the target oligonucleotide comprises using the nucleic acid primers. In some embodiments, a first adaptor strand and a second adaptor strand are independently ligated to each end of the target oligonucleotide, wherein the first adaptor strand comprises sequence complementary to one nucleic acid primer of the pair of nucleic acid primer and the second adaptor strand comprises sequence complementary to the other nucleic acid primer of the pair of nucleic acid primer. In some embodiments, the first adaptor strand comprises a reactive group, wherein the reactive group is configured to bind to the first chemically reactive group. In some embodiments, incubating the hydrogel particle and the second nucleic acid supramolecular structure is performed in a denaturing condition to avoid interaction between the adaptors and the nucleic acid primers. In some embodiments, prior to amplifying the target oligonucleotide, exchanging the denaturing condition to a non-denaturing condition may allow interaction between the adaptors and the nucleic acid primers. In some embodiments, amplifying the target oligonucleotide further comprises adding enzymes. In some embodiments, amplifying the target oligonucleotide further comprises using a thermocycler.

In some embodiments, the hydrogel particle is immobilized on a substrate. In some embodiments, the hydrogel particle is immobilized on the substrate by a binding between the second chemically reactive group disposed on the hydrogel particle and a corresponding binding molecule attached to the substrate, wherein the second chemically reactive group has a binding affinity to the corresponding binding molecule. In some embodiments, the second chemically reactive group comprises a thiol reactive group, an azide reactive group, an amine reactive group, or a carboxyl reactive group. In some embodiments, the substrate comprises a single-molecule array. In some embodiments, the single-molecule array comprises a second nucleic acid supramolecular structure, each second nucleic supramolecular structure comprising the corresponding binding molecule.

### Detection of an Analyte Molecule

The present disclosure also provides a method for detecting an analyte molecule. In some embodiments, the method comprises (i) incubating a first nucleic acid supramolecular structure, comprising a first chemically reactive group, with a plurality of polymer molecules to form a hydrogel matrix around the first nucleic acid supramolecular structure, (ii) providing a crosslinking agent to the hydrogel matrix around the first nucleic acid supramolecular structure to form the hydrogel particle, (iii) incubating the hydrogel particle with a second nucleic acid supramolecular structure, comprising the target oligonucleotide, to facilitate capture of the target oligonucleotide by the first chemically reactive group, (iv) providing conditions for amplification of the target oligonucleotide, and (v) detecting the analyte molecule based on lack of amplification from the step (iv).

In some embodiments, the first nucleic acid supramolecular structure is configured to be sufficiently thermostable to support a nucleic acid amplification. In some embodiments, the supramolecular structures may be thermostable up to about 80° C., about 85° C., about 90° C., about 95° C., about 100° C., about 105° C., about 110° C., about 115° C., about 120° C., or about 125° C. In some embodiments, the supramolecular structures may be thermostable at between about 80° C. and about 125° C., between about 85° C. and about 120° C., between about 90° C. and 115° C., between about 95° C. and about 110° C., or between about 100° C. and about 105° C. In some embodiments, the first chemically reactive group has an affinity to the target oligonucleotide. In some embodiments, the second supramolecular structure is configured to release the target oligonucleotide in absence of an analyte molecule.

In some embodiments, the first nucleic acid supramolecular structure further comprises the first core structure and a second chemically reactive group. In some embodiments, the first chemically reactive group is linked to the first core structure at a first location or a first set of locations. In some embodiments, the second chemically reactive group is linked to the first core structure at a second location, wherein the second location is spatially separated from the first location or the first set of locations. In some embodiments, the second chemically reactive group has an affinity to a corresponding binding molecule associated with a substrate. In some embodiments, the first location or the first set of locations and the second location are separated spatially enough to avoid cross-reactivity between the first chemically reactive group and the second chemically reactive group.

In some embodiments, the plurality of polymer molecules comprises a block copolymer comprising one or more charged regions, one or more uncharged regions, a free terminal associated with the uncharged region, and one or more reactive molecules attached to the free terminal. In some embodiments, the one or more uncharged regions comprises a neutral polymer. In some embodiments, the neutral polymer comprises polyethylene glycol (PEG). In some embodiments, the one or more charged region comprises a charged polymer. In some embodiments, the charged polymer comprises poly-L-lysine (PLL) or poly-acrylic acid (PAA).

In some embodiments, the second nucleic acid supramolecular structure further comprises a second core structure and a capture molecule, wherein the capture molecule is linked to the second core structure at a third location. In some embodiments, the target oligonucleotide is linked to the second core structure at a fourth location, wherein the fourth location is spatially separate from the third location. In some embodiments, the target oligonucleotide is configured to be released from the second core structure by providing a trigger to cleave the link between the target oligonucleotide and the second core structure in absence of the analyte molecule. In some embodiments, the target oligonucleotide remains to be bound to the second core structure through binding to the analyte molecule after providing the trigger to cleave the link between the target oligonucleotide and the second core structure in presence of the analyte molecule, wherein the analyte molecule also binds to the capture molecule at the third location of the second core structure.

In some embodiments, the analyte molecule may be detected based on whether copies of the target oligonucleotide are formed on the hydrogel particle. In some embodiments, the analyte molecule comprises a protein, a peptide, a peptide fragment, a lipid, a DNA, a RNA, an organic molecule, an inorganic molecule, complexes thereof, or any combinations thereof. In some embodiments, the trigger comprises DNA, RNA, a peptide, a small organic molecule, or combinations thereof. In some embodiments, the analyte molecule binds to the capture molecule through a chemical bond and/or binds to the target oligonucleotide through a chemical bond. In some embodiments, the capture molecule comprises a protein, a peptide, an antibody, an aptamer (RNA and DNA), a fluorophore, a darpin, a catalyst, a polymerization initiator, a polymer like PEG, or combinations thereof.

In some embodiments, the crosslinking agent comprises glutaraldehyde, bis(sulfosuccinimidyl)suberate (BS3), di-tert-butyl peroxide (DTBP), or combinations thereof. In some embodiments, nucleic acid primers are linked to the hydrogel matrix. In some embodiments, the nucleic acid primers facilitate amplification of the target oligonucleotide when the target oligonucleotide is captured by the first reactive group. In some embodiments, the first reactive group and the nucleic acid primers are extruded out of the hydrogel matrix. In some embodiments, the first nucleic acid supramolecular structure is configured to be thermostable via UV irradiation. In some embodiments, the first core structure and the second core structure independently comprise a deoxyribonucleic acid (DNA) origami, a ribonucleic acid (RNA) origami, a hybrid DNA/RNA origami, a single-stranded DNA origami, a single-stranded RNA origami, a hierarchically composed DNA and/or RNA origami, an enzymatically synthesized nucleic acid structure, a nucleic acid structure created by tile assembly, or combinations thereof.

In some embodiments, amplifying the target oligonucleotide comprises a local amplification. In some embodiments, amplifying the target oligonucleotide comprises using the nucleic acid primers. In some embodiments, a first adaptor strand and a second adaptor strand are independently ligated to each end of the target oligonucleotide, wherein the first adaptor strand comprises sequence complementary to one nucleic acid primer of the pair of nucleic acid primer and the second adaptor strand comprises sequence complementary to the other nucleic acid primer of the pair of nucleic acid primer. In some embodiments, the first adaptor strand comprises a reactive group, wherein the reactive group is configured to bind to the first chemically reactive group. In some embodiments, incubating the hydrogel particle and the second nucleic acid supramolecular structure is performed in a denaturing condition to avoid interaction between the adaptors and the nucleic acid primers. In some embodiments, prior to amplifying the target oligonucleotide, exchanging the denaturing condition to a non-denaturing condition may allow interaction between the adaptors and the nucleic acid primers. In some embodiments, amplifying the target oligonucleotide further comprises adding enzymes. In some embodiments, amplifying the target oligonucleotide further comprises using a thermocycler.

In some embodiments, the hydrogel particle is immobilized on a substrate. In some embodiments, the hydrogel particle is immobilized on the substrate by a binding between the second chemically reactive group disposed on the hydrogel particle and a corresponding binding molecule attached to the substrate, wherein the second chemically reactive group has a binding affinity to the corresponding binding molecule. In some embodiments, the second chemically reactive group comprises a thiol reactive group, an azide reactive group, an amine reactive group, or a carboxyl reactive group. In some embodiments, the substrate comprises a single-molecule array. In some embodiments, the single-molecule array comprises a second nucleic acid supramolecular structure, each second nucleic supramolecular structure comprising the corresponding binding molecule.

### Processing System

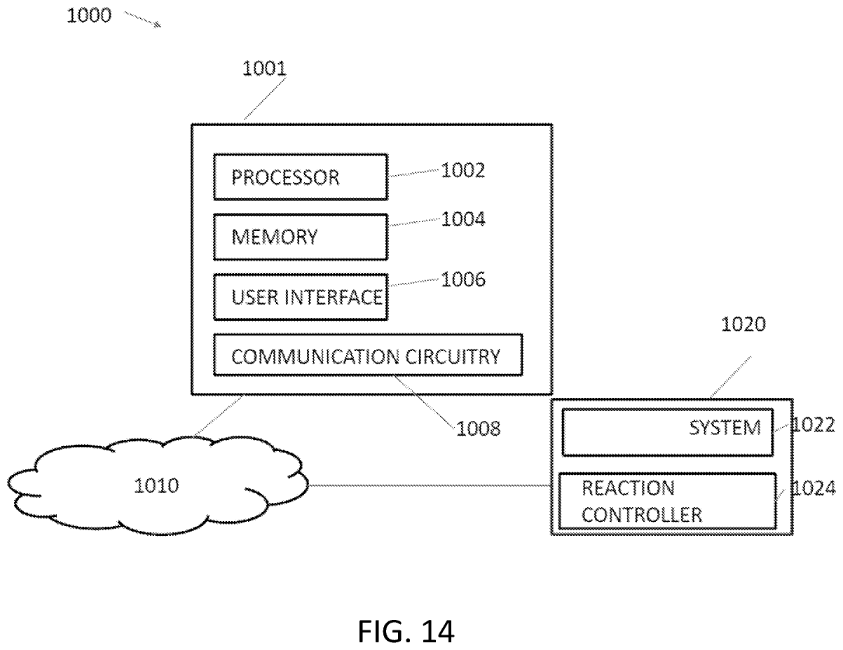

As may be appreciated, aspects of the presently described structures and techniques may be implemented or used if the further context of a device or system, such as an amplification or processing system 1000 as shown in FIG. 14. In particular, FIG. 14 shows an amplification or processing system 1000 that includes a controller 1001. The controller 1001 includes processor 1002 and a memory 1004 storing instructions configured to be executed by the processor 1002. The controller 1001 includes a user interface 1006 and communication circuitry 1008, e.g., to facilitate communication over the internet 1010 and/or over a wireless or wired network. The user interface 1006 facilitates user interaction with operational results or parameter specification as provided herein.

The processor 1002 is programmed to receive data and execute operational commands for performing one or more operations as described herein. The system 1000 also includes an amplification and/or imaging component 1020 that operates to control operations on or involving the hydrogel-coated particles 80 or capture and amplification structure 350 as discussed herein. A reaction controller 1024 may be present that controls sample incubation and appropriate release of reaction reagents, changes in buffer solution, and so forth at appropriate time points. The sensor 1022 may be one or more of an optical sensor (e.g., a fluorescent sensor, an infrared sensor), an image sensor, an electrical sensor, or a magnetic sensor. In an embodiment, the sensor 102 is a metal-oxide semiconductor image sensor device.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A hydrogel particle, comprising:
a first nucleic acid supramolecular structure including a first chemically reactive group having an affinity to a target oligonucleotide;
a hydrogel matrix formed around the first nucleic acid supramolecular structure;
a crosslinking agent to the hydrogel matrix disposed around the first nucleic acid supramolecular structure to form a hydrogel particle;
a plurality of primers attached to the hydrogel particle and configured to facilitate amplification of the target oligonucleotide; and
a second nucleic acid supramolecular structure comprising the target oligonucleotide and linked to the first chemically reactive group through the affinity of the first chemically reactive group to the target oligonucleotide;
wherein a link between the target oligonucleotide and the second supramolecular structure is configured to be cleaved by a trigger.

2. The hydrogel particle of claim 1, wherein the link between the target oligonucleotide and the second supramolecular structure is configured to be cleaved by the trigger in the absence of an analyte molecule.

3. The hydrogel particle of claim 2, wherein the link between the target oligonucleotide and the second supramolecular structure is configured to be cleaved by a deconstructor molecule.

4. The hydrogel particle of claim 2, further comprising the analyte molecule and a plurality of amplified copies of the target oligonucleotide attached to the primers.

5. The hydrogel particle of claim 1, wherein the link between the target oligonucleotide and the second supramolecular structure is configured to be cleaved by the trigger in the presence of an analyte molecule bound to the target oligonucleotide.

6. The hydrogel particle of claim 5, wherein the link between the target oligonucleotide and the second supramolecular structure is configured to be cleaved by a deconstructor molecule.

7. The hydrogel particle of claim 5, further comprising the analyte molecule, and wherein amplified copies of the target oligonucleotide are not generated.

8. The hydrogel particle of claim 5, further comprising a capture molecule linked to the second supramolecular structure and configured to bind to the analyte molecule such that in the presence of the analyte molecule, the target oligonucleotide is bound to the second supramolecular structure through binding of both the capture molecule and the target oligonucleotide to the analyte molecule.

9. The hydrogel particle of claim 8, further comprising the analyte molecule and a plurality of amplified copies of the target oligonucleotide attached to the primers.

10. The hydrogel particle of claim 1, wherein the first nucleic acid supramolecular structure further comprises a second chemically reactive group having an affinity to a binding molecule associated with a substrate.

11. A method for forming a monoclonal cluster of a target oligonucleotide on a hydrogel particle, comprising:
forming a hydrogel matrix around a first nucleic acid supramolecular structure comprising a first chemically reactive group having an affinity to a target oligonucleotide;
providing a crosslinking agent to the hydrogel matrix around the first nucleic acid supramolecular structure to form a hydrogel particle;
incubating the hydrogel particle with a second nucleic acid supramolecular structure comprising the target oligonucleotide, thereby facilitating capture of the target oligonucleotide by the first chemically reactive group;
providing a trigger to cleave a link between the target oligonucleotide and the second supramolecular structure; and
providing conditions for amplification of the target oligonucleotide.

12. The method of claim 11, wherein the trigger is configured to cleave the link between the target oligonucleotide and the second supramolecular structure in the absence of an analyte molecule.

13. The method of claim 12, wherein the trigger comprises a deconstructor molecule.

14. The method of claim 12, further comprising detecting the analyte molecule based on whether copies of the target oligonucleotide are formed on the hydrogel particle.

15. The method of claim 11, wherein the trigger is configured to cleave the link between the target oligonucleotide and the second supramolecular structure in the presence of an analyte molecule.

16. The method of claim 15, wherein the trigger comprises a deconstructor molecule.

17. The method of claim 15, further comprising detecting the analyte molecule based on lack of amplification of the target oligonucleotide.

18. The method of claim 15, wherein the second supramolecular structure comprises a capture molecule configured to bind to the analyte molecule, and wherein in the presence of the analyte molecule, the target oligonucleotide remains bound to the second supramolecular structure after providing the trigger, through binding of both the capture molecule and the target oligonucleotide to the analyte molecule.

19. The method of claim 18, further comprising detecting the analyte molecule based on whether copies of the target oligonucleotide are formed on the hydrogel particle.

20. The method of claim 11, wherein the first nucleic acid supramolecular structure further comprises a second chemically reactive group having an affinity to a binding molecule associated with a substrate.
